(19) United States
(12) Patent Application Publication
(10) Pub. No.: US 2025/0195477 A1
(43) Pub. Date: Jun. 19, 2025

(54) DEUTERATED IDAZOXAN AND METHODS OF USE THEREOF

(71) Applicant: Terran Biosciences Inc., Miami Beach, FL (US)

(72) Inventors: Samuel CLARK, Miami Beach, FL (US); Matthew DUNCTON, Las Vegas, NV (US)

(21) Appl. No.: 18/837,963

(22) PCT Filed: Feb. 16, 2023
(86) PCT No.: PCT/US2023/062767

§ 371 Date: Aug. 13, 2024

Related U.S. Application Data
(60) Provisional application No. 63/310,686, filed on Feb. 16, 2022.
(60) Provisional application No. 63/320,342, filed on Mar. 16, 2022.

(51) Int. Cl. A61K 31/4178; C07D 405/04

(52) U.S. Cl. CPC A61K 31/4178; C07D 405/04

(57) ABSTRACT

Deuterated forms of idazoxan according to Formula (I), and their pharmaceutically acceptable salts, pharmaceutical compositions containing these compounds, and methods of treatment or prevention using these compounds or pharmaceutical compositions are described. The compounds are useful for treating or preventing a disease or condition selected from psychosis, schizophrenia, schizoaffective disorder, Parkinson's disease, Lewy body dementia, sleep disorder (including insomnia), agitation, mood disorder (including depression), thromboembolic disorder, autism, and attention deficit hyperactivity disorder.

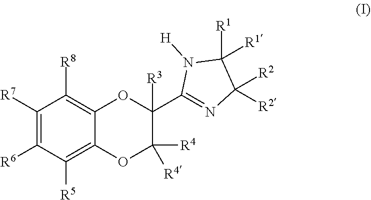

### CROSS-REFERENCE TO RELATED APPLICATIONS

This application claims priority to, and the benefit of, U.S. Provisional Application No. 63/320,342, filed Mar. 16, 2022, and U.S. Provisional Application No. 63/310,686, filed Feb. 16, 2022, the contents of each of which are incorporated by reference in their entirety.

### BACKGROUND

Idazoxan has been evaluated as both a selective α2 adrenergic receptor antagonist, and an antagonist for the imidazoline receptor but suffers from conversion to one or more metabolites. Also, only about 27-28% of idazoxan reaches systemic circulation, being cleared from the systemic circulation via the urine. The major metabolites of idazoxan include hydroxylation of the benzene ring at the 6-position and/or 7-position along with their glucuronide conjugates. A sulfate conjugate of the 7-OH metabolite can also be produced. Only small amounts of the 5-OH compound were detected and there is no evidence for the 8-OH compound being a metabolite of idazoxan. With respect to the imidazoline ring, the major metabolite resulted from ring opening. There are a number of the other minor metabolites which have not yet been identified. A scheme for the metabolism of idazoxan is given below:

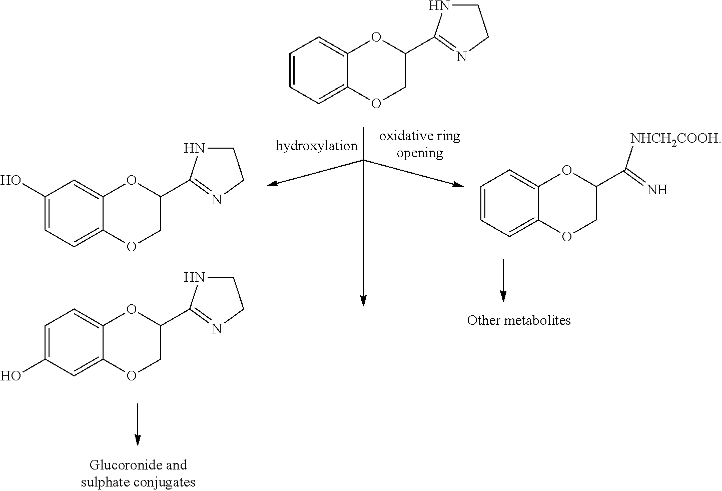

The present disclosure provides deuterated idazoxan analogs designed to improve the pharmacokinetic and metabolic properties of idazoxan. Indeed, a potentially attractive strategy for improving a drug's metabolic properties is deuterium modification In this approach, one attempts to slow the metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon.

Over the past 35 years, the effects of deuterium substitution on the rate of metabolism have been reported for a very small percentage of approved drugs (see, e.g., Blake, M I, et al., J Pharm Sci, 1975, 64:367-91; Foster, AB, Adv Drug Res 1985, 14:1-40 (“Foster”); Kushner, D J, et al., Can J Physiol Pharmacol 1999, 79-88; Fisher, M B, et al., Curr Opin Drug Discov Devel, 2006, 9:101-09 (“Fisher”)). The results have been variable and unpredictable. For some compounds, deuteration caused decreased metabolic clearance in vivo. For others, there was no change in metabolism. Still others demonstrated increased metabolic clearance. The variability in effects of deuteration has also led experts to question or dismiss deuterium modification as a viable drug design strategy for inhibiting adverse metabolism (see Foster at p. 35 and Fisher at p. 101)

### SUMMARY

In one aspect, the present application relates to a compound of Formula (I):

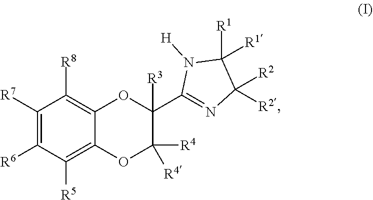

or a pharmaceutically acceptable salt, solvate, hydrate, tautomer, or stereoisomer thereof, wherein R¹, R¹′, R², R²′, R³, R⁴, R⁴′, R⁵, R⁶, R⁷, and R⁸ are each independently selected from hydrogen and deuterium, provided that at least one of R¹, R¹′, R², R²′, R³, R⁴, R⁴′, R⁵, R⁶, R⁷, and R⁸ is deuterium.

In another aspect, the present application relates to a compound of Formula (II) or Formula (III):

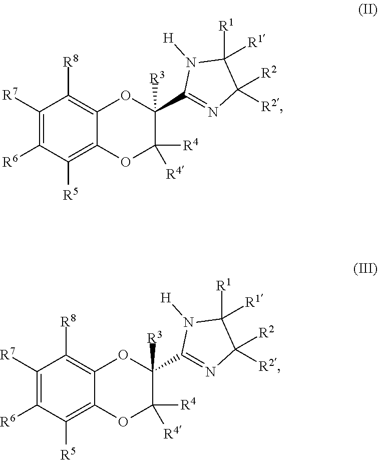

or a pharmaceutically acceptable salt, solvate, hydrate, tautomer, or stereoisomer thereof, wherein R¹, R¹′, R², R²′, R³, R⁴, R⁴′, R⁵, R⁶, R⁷, and R⁸ are each independently selected from hydrogen and deuterium, provided that at least one of R¹, R¹′, R², R²′, R³, R⁴, R⁴′, R⁵, R⁶, R⁷, and R⁸ is deuterium.

In another aspect, the present application relates to a compound of Formula (II-A) or Formula (III-A):

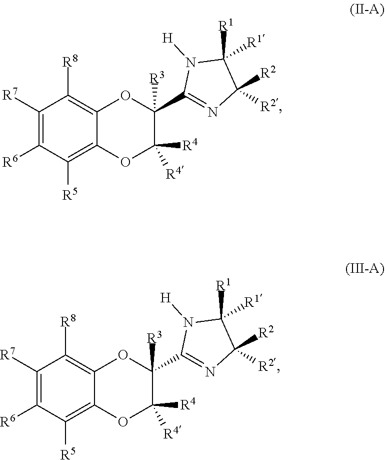

or a pharmaceutically acceptable salt, solvate, hydrate, tautomer, or stereoisomer thereof, wherein R¹, R¹′, R², R²′, R³, R⁴, R⁴′, R⁵, R⁶, R⁷, and R⁸ are each independently selected from hydrogen and deuterium, provided that at least one of R¹, R¹′, R², R²′, R³, R⁴, R⁴′, R⁵, R⁶, R⁷, and R⁸ is deuterium.

In another aspect, the present application relates to a compound of Formula (I-A):

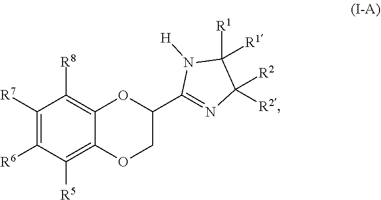

or a pharmaceutically acceptable salt, solvate, hydrate, tautomer, or stereoisomer thereof, wherein R¹, R¹′, R², R²′, R⁵, R⁶, R⁷, and R⁸ are each independently selected from hydrogen and deuterium, provided that at least one of R¹, R¹′, R², R²′, R⁵, R⁶, R⁷, and R⁸ is deuterium.

In another aspect, the present application relates to a pharmaceutical composition comprising a therapeutically effective amount of a compound of the application, or a pharmaceutically acceptable salt, solvate, hydrate, tautomer, or stereoisomer thereof, and a pharmaceutically acceptable carrier.

Another aspect of the present application relates to a method of treating a neurological condition. The method comprises administering to a subject in need thereof an effective amount of a compound of the application or a pharmaceutically acceptable salt, solvate, hydrate, tautomer, or stereoisomer thereof, or a pharmaceutical composition of the application.

Another aspect of the present application relates to a method of treating or preventing a disease, wherein the diseases is caused by a neurological condition. The method comprises administering to a subject in need thereof an effective amount of a compound of the application or a pharmaceutically acceptable salt, solvate, hydrate, tautomer, or stereoisomer thereof, or a pharmaceutical composition of the application.

Another aspect of the present application relates to a kit comprising a compound of the application or a pharmaceutically acceptable salt, solvate, hydrate, tautomer, or stereoisomer thereof, or a pharmaceutical composition of the application.

Another aspect of the present application relates to a compound of the application or a pharmaceutically acceptable salt, solvate, hydrate, tautomer, or stereoisomer thereof, or a pharmaceutical composition of the application, for use in the manufacture of a medicament for treating a neurological condition.

The details of the disclosure are set forth in the accompanying description below. Although methods and materials similar or equivalent to those described herein can be used in the practice or testing of the present application, illustrative methods and materials are now described. In the case of conflict, the present specification, including definitions, will control. In addition, the materials, methods, and examples are illustrative only and are not intended to be limiting. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

The contents of all references (including literature references, issued patents, published patent applications, and co-pending patent applications) cited throughout this application are hereby expressly incorporated herein in their entireties by reference. The references cited herein are not admitted to be prior art to the application.

### DETAILED DESCRIPTION

In one aspect, the present application relates to a compound of Formula (I):

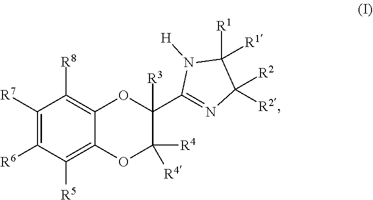

or a pharmaceutically acceptable salt, solvate, hydrate, tautomer, or stereoisomer thereof, wherein R¹, R¹′, R², R²′, R³, R⁴, R⁴′, R⁵, R⁶, R⁷, and R⁸ are each independently selected from hydrogen and deuterium, provided that at least one of R¹, R¹′, R², R²′, R³, R⁴, R⁴′, R⁵, R⁶, R⁷, and R⁸ is deuterium.

In another aspect, the present application relates to a compound of Formula (II) or Formula (III):

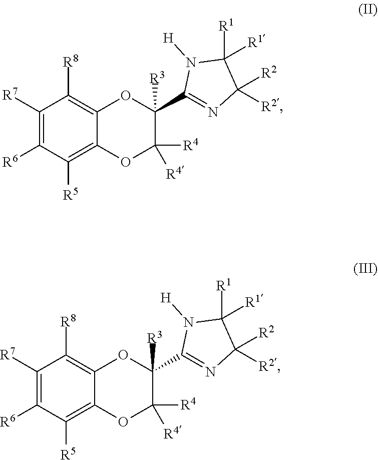

or a pharmaceutically acceptable salt, solvate, hydrate, tautomer, or stereoisomer thereof, wherein R¹, R¹′, R², R²′, R³, R⁴, R⁴′, R⁵, R⁶, R⁷, and R⁸ are each independently selected from hydrogen and deuterium, provided that at least one of R¹, R¹′, R², R²′, R³, R⁴, R⁴′, R⁵, R⁶, R⁷, and R⁸ is deuterium.

In another aspect, the present application relates to a compound of Formula (II-A) or Formula (III-A):

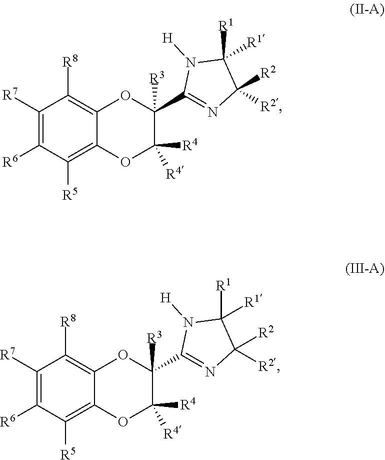

or a pharmaceutically acceptable salt, solvate, hydrate, tautomer, or stereoisomer thereof, wherein R¹, R¹′, R², R²′, R³, R⁴, R⁴′, R⁵, R⁶, R⁷, and R⁸ are each independently selected from hydrogen and deuterium, provided that at least one of R¹, R¹′, R², R²′, R³, R⁴, R⁴′, R⁵, R⁶, R⁷, and R⁸ is deuterium.

In another aspect, the present application relates to a compound of Formula (I-A):

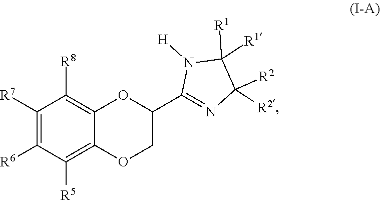

or a pharmaceutically acceptable salt, solvate, hydrate, tautomer, or stereoisomer thereof, wherein R¹, R¹′, R², R²′, R⁵, R⁶, R⁷, and R⁸ are each independently selected from hydrogen and deuterium, provided that at least one of R¹, R¹′, R², R²′, R⁵, R⁶, R⁷, and R⁸ are deuterium.

In another aspect, the present application relates to a compound of Formula (I-A-R) or (1-A-S):

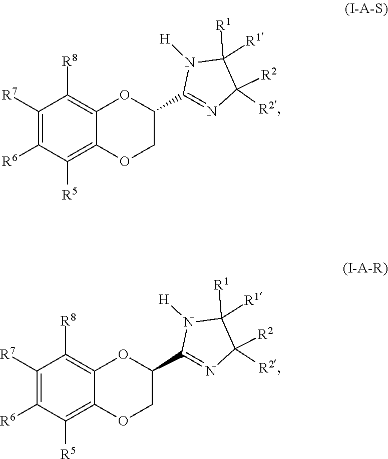

or a pharmaceutically acceptable salt, solvate, hydrate, tautomer, or stereoisomer thereof, wherein R¹, R¹′, R², R²′, R⁵, R⁶, R⁷, and R⁸ are each independently selected from hydrogen and deuterium, provided that at least one of R¹, R¹′, R², R²′, R⁵, R⁶, R⁷, and R⁸ is deuterium.

In one aspect, the present application relates to a compound of Formula (IV), Formula (IV-S), or Formula (IV-R):

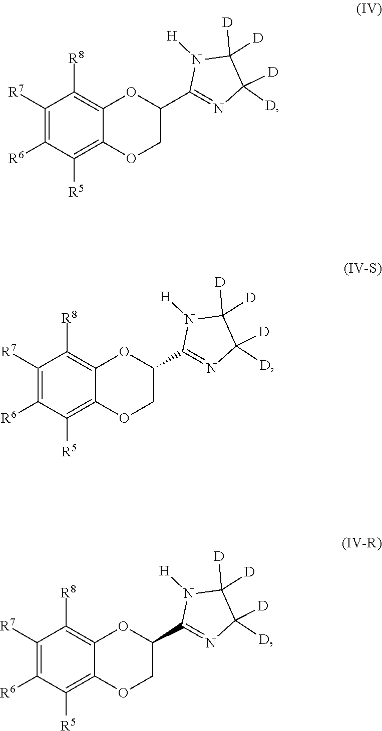

or a pharmaceutically acceptable salt, solvate, hydrate, tautomer, or stereoisomer thereof, wherein R⁵, R⁶, R⁷, and R⁸ are each independently selected from hydrogen and deuterium.

In one aspect, the present application relates to a compound of Formula (V), Formula (V-S), or Formula (V-R):

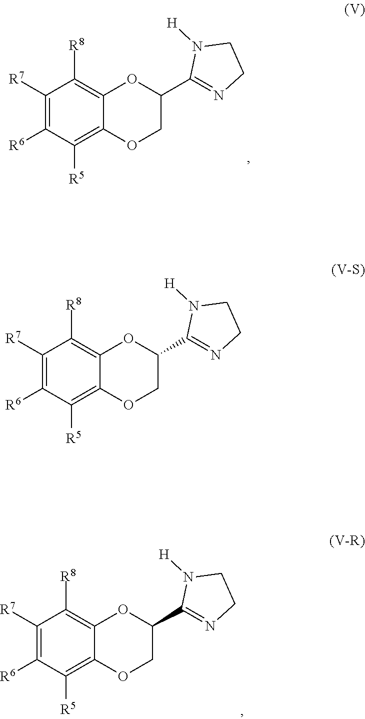

or a pharmaceutically acceptable salt, solvate, hydrate, tautomer, or stereoisomer thereof, wherein R⁵, R⁶, R⁷, and R⁸ are each independently selected from hydrogen and deuterium provided that at least one of R⁵, R⁶, R⁷, and R⁸ are each independently selected from hydrogen and deuterium.

In one aspect, the present application relates to a compound of Formula (VI), Formula (VI-S), or Formula (VI-R):

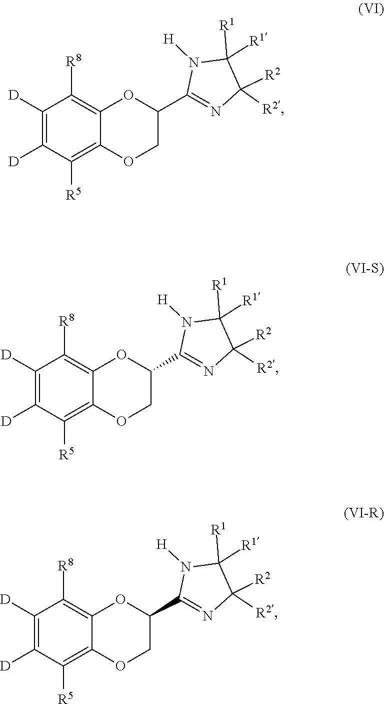

or a pharmaceutically acceptable salt, solvate, hydrate, tautomer, or stereoisomer thereof, wherein R¹, R¹, R², R²′, R⁵, and R⁸ are each independently selected from hydrogen and deuterium.

In one aspect, the present application relates to a compound of Formula (VII), Formula (VII-S), or Formula (VII-R):

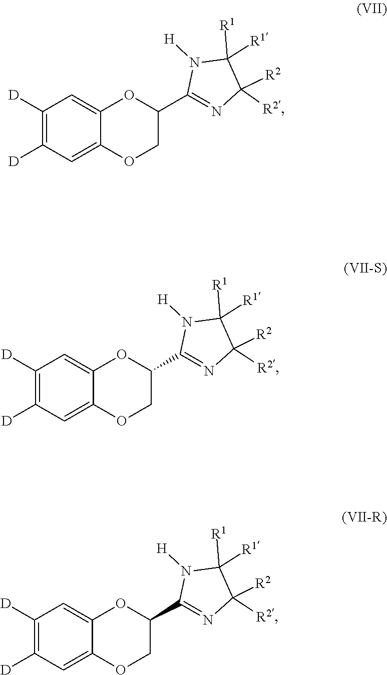

or a pharmaceutically acceptable salt, solvate, hydrate, tautomer, or stereoisomer thereof, wherein R¹, R¹, R², and R²′ are each independently selected from hydrogen and deuterium.

In one aspect, the present application relates to a compound of Formula (VIII), Formula (VIII-S), or Formula (VIII-R):

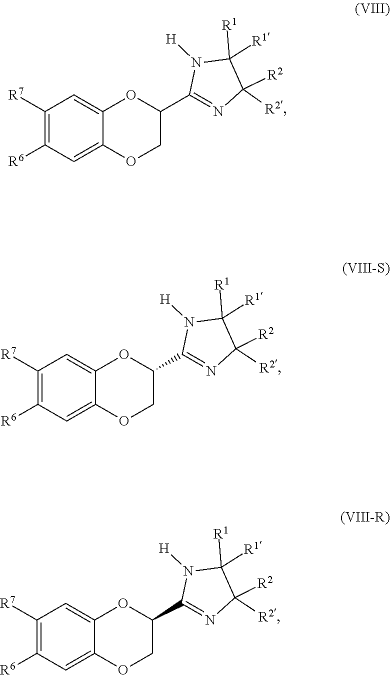

or a pharmaceutically acceptable salt, solvate, hydrate, tautomer, or stereoisomer thereof, wherein R¹, R¹, R², R²′, R⁶, and R⁷ are each independently selected from hydrogen and deuterium provided that at least one of R¹, R¹, R², R²′, R⁶, and R⁷ are each independently selected from hydrogen and deuterium.

In one aspect, the present application relates to a compound of Formula (IX), Formula (IX-S), or Formula (IX-R):

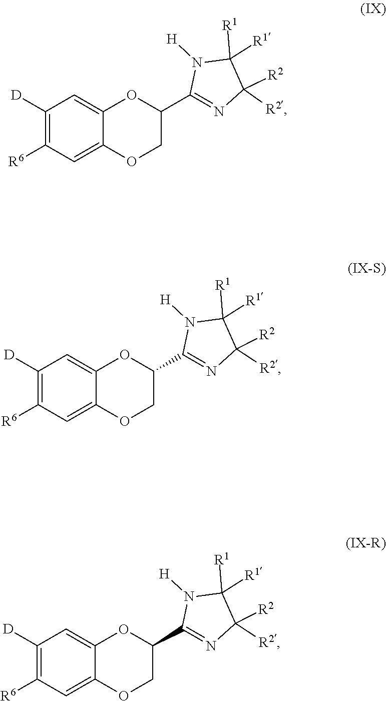

or a pharmaceutically acceptable salt, solvate, hydrate, tautomer, or stereoisomer thereof, wherein R¹, R¹, R², R²′, and R⁶ are each independently selected from hydrogen and deuterium.

In one aspect, the present application relates to a compound of Formula (X), Formula (X-S), or Formula (X-R):

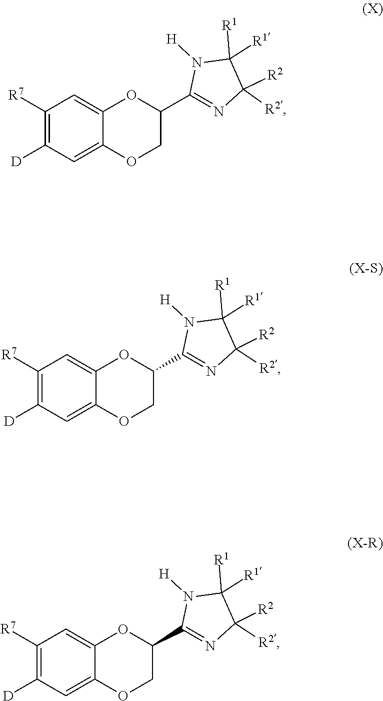

or a pharmaceutically acceptable salt, solvate, hydrate, tautomer, or stereoisomer thereof, wherein R¹, R¹, R², R²′, and R⁷ are each independently selected from hydrogen and deuterium.

In one aspect, the present application relates to a compound of Formula (XI), Formula

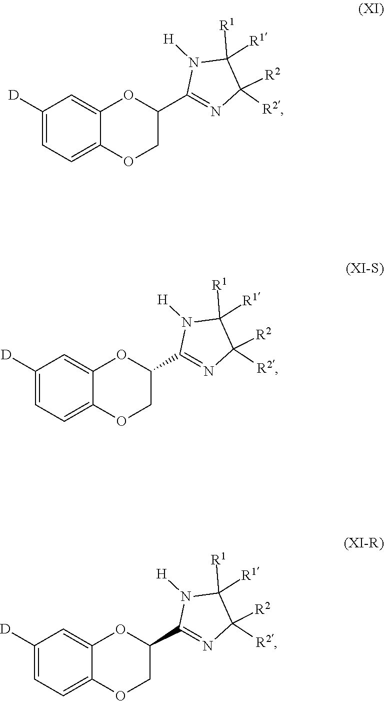

or a pharmaceutically acceptable salt, solvate, hydrate, tautomer, or stereoisomer thereof, wherein R¹, R¹, R², and R²′ are each independently selected from hydrogen and deuterium.

In one aspect, the present application relates to a compound of Formula (XII), Formula (XII-S), or Formula (XII-R):

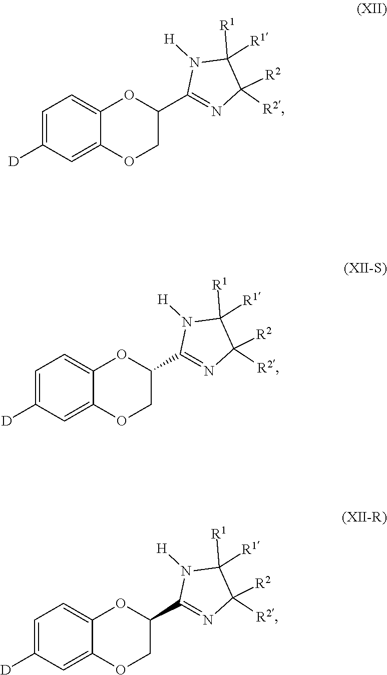

or a pharmaceutically acceptable salt, solvate, hydrate, tautomer, or stereoisomer thereof, wherein R¹, R¹, R², and R²′ are each independently selected from hydrogen and deuterium.

In one aspect, the present application relates to a compound of Formula (I), Formula (II), Formula (III), Formula (II-A), Formula (III-A), Formula (I-A), Formula (I-A-R), Formula (I-A-S), Formula (IV), Formula (IV-S), Formula (IV-R), Formula (V), Formula (V-S), Formula (V-R), Formula (VI), Formula (VI-S), Formula (VI-R), Formula (VII), Formula (VII-S), Formula (VII-R), Formula (VIII), Formula (VIII-S), Formula (VIII-R), Formula (IX), Formula (IX-S), Formula (IX-R), Formula (X), Formula (X-S), Formula (X-R), Formula (XI), Formula (XI-S), Formula (XI-R), Formula (XII), Formula (XII-S), or Formula (XII-R) or a pharmaceutically acceptable salt thereof. In one aspect, the present application relates to a compound of Formula (I), Formula (II), Formula (III), Formula (II-A), Formula (III-A), Formula (I-A), Formula (I-A-R), Formula (I-A-S), Formula (IV), Formula (IV-S), Formula (IV-R), Formula (V), Formula (V-S), Formula (V-R), Formula (VI), Formula (VI-S), Formula (VI-R), Formula (VII), Formula (VII-S), Formula (VII-R), Formula (VIII), Formula (VIII-S), Formula (VIII-R), Formula (IX), Formula (IX-S), Formula (IX-R), Formula (X), Formula (X-S), Formula (X-R), Formula (XI), Formula (XI-S), Formula (XI-R), Formula (XII), Formula (XII-S), or Formula (XII-R).

In some embodiments, one of R¹, R¹′, R², R²′, R³, R¹, R⁴′, R⁵, R⁶, R⁷, and R⁸ is deuterium.

In some embodiments, one of R¹, R¹′, R², R²′, R⁵, R⁶, R⁷, and R⁸ is deuterium.

In some embodiments, R¹ is deuterium.

In some embodiments, R¹′ is deuterium.

In some embodiments, R² is deuterium.

In some embodiments, R²′ is deuterium.

In some embodiments, R³ is deuterium.

In some embodiments, R⁴ is deuterium.

In some embodiments, R⁴′ is deuterium.

In some embodiments, R⁵ is deuterium.

In some embodiments, R⁶ is deuterium.

In some embodiments, R⁷ is deuterium.

In some embodiments, R⁸ is deuterium.

In some embodiments, two of R¹, R¹′, R², R²′, R³, R⁴, R⁴′, R⁵, R⁶, R⁷, and R⁸ are deuterium.

In some embodiments, two of R¹, R¹′, R², R²′, R⁵, R⁶, R⁷, and R⁸ are deuterium.

In some embodiments, R¹ is deuterium and R¹′ is deuterium.

In some embodiments, R² is deuterium and R²′ is deuterium.

In some embodiments, R⁴ is deuterium and R⁴′ is deuterium.

In some embodiments, R⁵ is deuterium and R⁶ is deuterium.

In some embodiments, R⁵ is deuterium and R⁷ is deuterium.

In some embodiments, R⁵ is deuterium and R⁸ is deuterium.

In some embodiments, R⁶ is deuterium and R⁷ is deuterium.

In some embodiments, R⁶ is deuterium and R⁸ is deuterium.

In some embodiments, R⁷ is deuterium and R⁸ is deuterium.

In some embodiments, three of R¹, R¹′, R², R²′, R³, R⁴, R⁴′, R⁵, R⁶, R⁷, and R⁸ are deuterium.

In some embodiments, three of R¹, R¹′, R², R²′, R⁵, R⁶, R⁷, and R⁸ are deuterium.

In some embodiments, R¹, R¹′, and R⁵ are each deuterium.

In some embodiments, R¹, R¹′, and R⁶ are each deuterium.

In some embodiments, R¹, R¹′, and R⁷ are each deuterium.

In some embodiments, R¹, R¹′, and R⁸ are each deuterium.

In some embodiments, R², R²′, and R⁵ are each deuterium.

In some embodiments, R², R²′, and R⁶ are each deuterium.

In some embodiments, R², R²′, and R⁷ are each deuterium.

In some embodiments, R², R²′, and R⁸ are each deuterium.

In some embodiments, R¹, R⁵ and R⁶ are each deuterium.

In some embodiments, R¹, R⁵ and R⁷ are each deuterium.

In some embodiments, R¹, R⁵ and R⁸ are each deuterium.

In some embodiments, R¹, R⁶ and R⁷ are each deuterium.

In some embodiments, R¹, R⁶ and R⁸ are each deuterium.

In some embodiments, R¹, R⁷ and R⁸ are each deuterium.

In some embodiments, R¹′, R⁵ and R⁶ are each deuterium.

In some embodiments, R¹′, R⁵ and R⁷ are each deuterium.

In some embodiments, R¹′, R⁵ and R⁸ are each deuterium.

In some embodiments, R¹′, R⁶ and R⁷ are each deuterium.

In some embodiments, R¹′, R⁶ and R⁸ are each deuterium.

In some embodiments, R¹′, R⁷ and R⁸ are each deuterium.

In some embodiments, R², R⁵ and R⁶ are each deuterium.

In some embodiments, R², R⁵ and R⁷ are each deuterium.

In some embodiments, R², R⁵ and R⁸ are each deuterium.

In some embodiments, R², R⁶ and R⁷ are each deuterium.

In some embodiments, R², R⁶ and R⁸ are each deuterium.

In some embodiments, R², R⁷ and R⁸ are each deuterium.

In some embodiments, R²′, R⁵ and R⁶ are each deuterium.

In some embodiments, R²′, R⁵ and R⁷ are each deuterium.

In some embodiments, R²′, R⁵ and R⁸ are each deuterium.

In some embodiments, R²′, R⁶ and R⁷ are each deuterium.

In some embodiments, R²′, R⁶ and R⁸ are each deuterium.

In some embodiments, R²′, R⁷ and R⁸ are each deuterium.

In some embodiments, four of R¹, R¹′, R², R²′, R³, R¹, R⁴′, R⁵, R⁶, R⁷, and R⁸ are deuterium.

In some embodiments, four of R¹, R¹′, R², R²′, R⁵, R⁶, R⁷, and R⁸ are deuterium.

In some embodiments, R¹, R¹′, R⁵ and R⁶ are each deuterium.

In some embodiments, R¹, R¹′, R⁵ and R⁷ are each deuterium.

In some embodiments, R¹, R¹′, R⁵ and R⁸ are each deuterium.

In some embodiments, R¹, R¹′, R⁶ and R⁷ are each deuterium.

In some embodiments, R¹, R¹′, R⁶ and R⁸ are each deuterium.

In some embodiments, R¹, R¹′, R⁷ and R⁸ are each deuterium.

In some embodiments, R², R²′, R⁵ and R⁶ are each deuterium.

In some embodiments, R², R²′, R⁵ and R⁷ are each deuterium.

In some embodiments, R², R²′, R⁵ and R⁸ are each deuterium.

In some embodiments, R², R²′, R⁶ and R⁷ are each deuterium.

In some embodiments, R², R²′, R⁶ and R⁸ are each deuterium.

In some embodiments, R², R²′, R⁷ and R⁸ are each deuterium.

In some embodiments, R⁵, R⁶, R⁷, and R⁸ are each deuterium.

In some embodiments, five of R¹, R¹′, R², R²′, R³, R⁴, R⁴′, R⁵, R⁶, R⁷, and R⁸ are deuterium.

In some embodiments, five of R¹, R¹′, R², R²′, R⁵, R⁶, R⁷, and R⁸ are deuterium.

In some embodiments, R¹, R⁵, R⁶, R⁷, and R⁸ are each deuterium.

In some embodiments, R¹′, R⁵, R⁶, R⁷, and R⁸ are each deuterium.

In some embodiments, R², R⁵, R⁶, R⁷, and R⁸ are each deuterium.

In some embodiments, R²′, R⁵, R⁶, R⁷, and R⁸ are each deuterium.

In some embodiments, six of R¹, R¹′, R², R²′, R³, R⁴, R⁴′, R⁵, R⁶, R⁷, and R⁸ are deuterium.

In some embodiments, six of R¹, R¹′, R², R²′, R⁵, R⁶, R⁷, and R⁸ are deuterium.

In some embodiments, R¹, R¹′, R⁵, R⁶, R⁷, and R⁸ are each deuterium.

In some embodiments, R², R²′, R⁵, R⁶, R⁷, and R⁸ are each deuterium.

In some embodiments, R¹, R², R⁵, R⁶, R⁷, and R⁸ are each deuterium.

In some embodiments, R¹, R²′, R⁵, R⁶, R⁷, and R⁸ are each deuterium.

In some embodiments, R¹′, R², R⁵, R⁶, R⁷, and R⁸ are each deuterium.

In some embodiments, R¹′, R²′, R⁵, R⁶, R⁷, and R⁸ are each deuterium.

In some embodiments, seven of R¹, R¹′, R², R²′, R³, R⁴, R⁴′, R⁵, R⁶, R⁷, and R⁸ are deuterium.

In some embodiments, seven of R¹, R¹′, R², R²′, R⁵, R⁶, R⁷, and R⁸ are deuterium.

In some embodiments, R¹, R¹′, R², R⁵, R⁶, R⁷, and R⁸ are each deuterium.

In some embodiments, R¹, R¹′, R²′, R⁵, R⁶, R⁷, and R⁸ are each deuterium.

In some embodiments, R¹, R², R²′, R⁵, R⁶, R⁷, and R⁸ are each deuterium.

In some embodiments, R¹′, R², R²′, R⁵, R⁶, R⁷, and R⁸ are each deuterium.

In some embodiments, eight of R¹, R¹′, R², R²′, R³, R⁴, R⁴′, R⁵, R⁶, R⁷, and R⁸ are deuterium.

In some embodiments, eight of R¹, R¹′, R², R²′, R⁵, R⁶, R⁷, and R⁸ are deuterium.

In some embodiments, R¹, R¹′, R², R²′, R⁵, R⁶, R⁷, and R⁸ are each deuterium.

In some embodiments, at least two of R¹, R¹′, R², R²′, R³, R⁴, R⁴′, R⁵, R⁶, R⁷, and R⁸ are deuterium.

In some embodiments, at least three of R¹, R¹′, R², R²′, R³, R⁴, R⁴′, R⁵, R⁶, R⁷, and R⁸ are deuterium.

In some embodiments, at least four of R¹, R¹′, R², R²′, R³, R⁴, R⁴′, R⁵, R⁶, R⁷, and R⁸ are deuterium.

In some embodiments, at least five of R¹, R¹′, R², R²′, R³, R⁴, R⁴′, R⁵, R⁶, R⁷, and R⁸ are deuterium.

In some embodiments, at least six of R¹, R¹′, R², R²′, R³, R⁴, R⁴′, R⁵, R⁶, R⁷, and R⁸ are deuterium.

In some embodiments, at least seven of R¹, R¹′, R², R²′, R³, R⁴, R⁴′, R⁵, R⁶, R⁷, and R⁸ are deuterium.

In some embodiments, at least eight of R¹, R¹′, R², R²′, R³, R⁴, R⁴′, R⁵, R⁶, R⁷, and R⁸ are deuterium.

In some embodiments, at least nine of R¹, R¹′, R², R²′, R³, R⁴, R⁴′, R⁵, R⁶, R⁷, and R⁸ are deuterium.

In some embodiments, at least ten of R¹, R¹′, R², R²′, R³, R¹, R⁴′, R⁵, R⁶, R⁷, and R⁸ are deuterium.

In some embodiments, all of R¹, R¹′, R², R²′, R³, R⁴, R⁴′, R⁵, R⁶, R⁷, and R⁸ are deuterium.

In some embodiments, where a variable is designated as deuterium, the remainder of the variables are hydrogen. In some embodiments, where a variable is designated as deuterium, the remainder of the variables are either hydrogen or deuterium.

Unless otherwise stated, when a position is designated specifically as “H” or “hydrogen”, the position is understood to have hydrogen or its isotopes, such as deuterium (d, d, D, or ²H) or tritium (3H) at its natural abundance isotopic composition.

In some embodiments, each position designated as deuterium has an abundance of deuterium at that position that is substantially greater than the natural abundance of deuterium, which is about 0.015%.

In some embodiments, each position designated as deuterium has at least about 50%, at least about 60%, at least about 65%, at least about 70%, at least about 75%, at least about 80%, at least about 85%, at least about 90%, at least about 95%, or at least about 99% incorporation of deuterium.

In some embodiments, each position designated as deuterium has at least about 90%, at least about 91%, at least about 92%, at least about 93%, at least about 94%, at least about 95%, at least about 96%, at least about 97%, at least about 98%, or at least about 99% incorporation of deuterium.

In some embodiments, each position designated specifically as deuterium has at least about 90% incorporation of deuterium. In some embodiments, each position designated specifically as deuterium has at least about 95% incorporation of deuterium. In some embodiments, each position designated specifically as deuterium has at least about 97% incorporation of deuterium.

Any of the embodiments herein apply to Formula (I), Formula (II), Formula (III) Formula (II-A), Formula (III-A), Formula (I-A), Formula (I-A-R), Formula (I-A-S), Formula (IV), Formula (IV-S), Formula (IV-R), Formula (V), Formula (V-S), Formula (V-R), Formula (VI), Formula (VI-S), Formula (VI-R), Formula (VII), Formula (VII-S), Formula (VII-R), Formula (VIII), Formula (VIII-S), Formula (VIII-R), Formula (IX), Formula (IX-S), Formula (IX-R), Formula (X), Formula (X-S), Formula (X-R), Formula (XI), Formula (XI-S), Formula (XI-R), Formula (XII), Formula (XII-S), Formula (XII-R), or Table 1 where applicable. Any of the groups described above for any of R¹, R¹′, R², R²′, R³, R⁴, R⁴′, R⁵, R⁶, R⁷, and R⁸ can be combined with any of the groups described above for the remainder of R¹, R¹′, R², R²′, R³, R⁴, R⁴′, R⁵, R⁶, R⁷, and R⁸ where applicable.

Representative compounds of the present application are shown in Table 1.

TABLE 1
Compounds of the Present Disclosure

| ID No. | Chemical Structure | IUPAC Name |
|---|---|---|
| 1 | 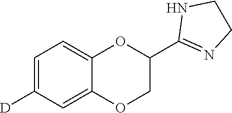 [SMILES: [2H]c1ccc2c(c1)OCC(C1=NCCN1)O2] | 2-(2,3-dihydrobenzo[b][1,4]dioxin-2-yl-6- d)-4,5-dihydro-1H-imidazole |
| 1-R | 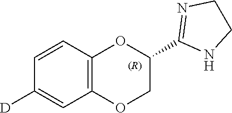 | (R)-2-(2,3-dihydrobenzo[b][1,4]dioxin-2-yl- 6-d)-4,5-dihydro-1H-imidazole |
| 1-S | 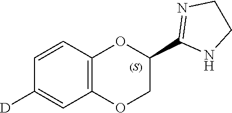 | (S)-2-(2,3-dihydrobenzo[b][1,4]dioxin-2-yl- 6-d)-4,5-dihydro-1H-imidazole |
| 2 | 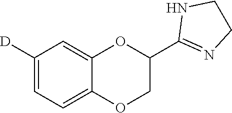 [SMILES: [2H]c1ccc2c(c1)OC(C1=NCCN1)CO2] | 2-(2,3-dihydrobenzo[b][1,4]dioxin-2-yl-7- d)-4,5-dihydro-1H-imidazole |
| 2-R | 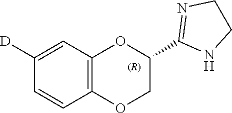 | (R)-2-(2,3-dihydrobenzo[b][1,4]dioxin-2-yl- 7-d)-4,5-dihydro-1H-imidazole |
| 2-S | 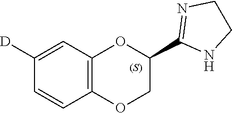 | (S)-2-(2,3-dihydrobenzo[b][1,4]dioxin-2-yl- 7-d)-4,5-dihydro-1H-imidazole |
| 3 | 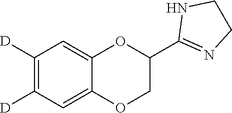 [SMILES: [2H]c1cc2c(cc1[2H])OC(C1=NCCN1)CO2] | 2-(2,3-dihydrobenzo[b][1,4]dioxin-2-y1-6,7- d2)-4,5-dihydro-1H-imidazole |
| 3-R | 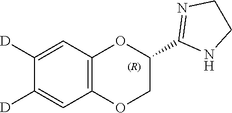 | (R)-2-(2,3-dihydrobenzo[b][1,4]dioxin-2-yl- 6,7-d2)-4,5-dihydro-1H-imidazole |
| 3-S | 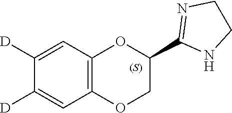 | (S)-2-(2,3-dihydrobenzo[b][1,4]dioxin-2-yl- 6,7-d2)-4,5-dihydro-1H-imidazole |
| 4 | 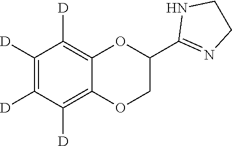 [SMILES: [2H]c1c([2H])c([2H])c2c(c1[2H])OCC(C1=NCCN1)O2] | 2-(2,3-dihydrobenzo[b][1,4]dioxin-2-yl- 5,6,7,8-d4)-4,5-dihydro-1H-imidazole |
| 4-R | 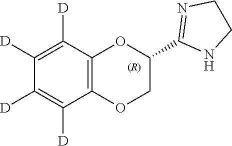 | (R)-2-(2,3-dihydrobenzo[b][1,4]dioxin-2-yl- 5,6,7,8-d4)-4,5-dihydro-1H-imidazole |
| 4-S | 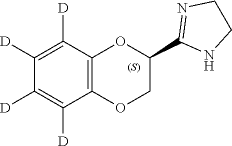 | (S)-2-(2,3-dihydrobenzo[b][1,4]dioxin-2-yl- 5,6,7,8-d4)-4,5-dihydro-1H-imidazole |
| 5 | 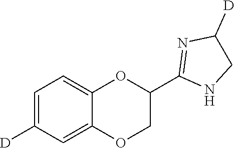 [SMILES: [2H]c1ccc2c(c1)OCC(C1=NC([2H])CN1)O2] | 2-(2,3-dihydrobenzo[b][1,4]dioxin-2-yl-6- d)-4,5-dihydro-1H-imidazole-4-d |
| 6 | 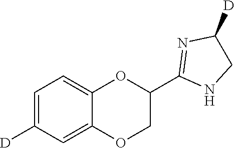 [SMILES: [2H]c1ccc2c(c1)OCC(C1=N[C@@H]([2H])CN1)O2] | (4S)-2-(2,3-dihydrobenzo[b][1,4]dioxin-2- yl-6-d)-4,5-dihydro-1H-imidazole-4-d |
| 7 | 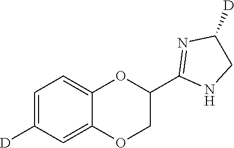 [SMILES: [2H]c1ccc2c(c1)OCC(C1=N[C@H]([2H])CN1)O2] | (4R)-2-(2,3-dihydrobenzo[b][1,4]dioxin-2- yl-6-d)-4,5-dihydro-1H-imidazole-4-d |
| 8 | 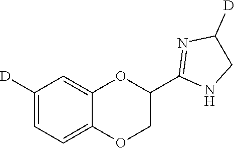 [SMILES: [2H]c1ccc2c(c1)OC(C1=NC([2H])CN1)CO2] | 2-(2,3-dihydrobenzo[b][1,4]dioxin-2-yl-7- d)-4,5-dihydro-1H-imidazole-4-d |
| 9 | 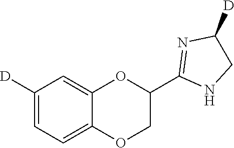 [SMILES: [2H]c1ccc2c(c1)OC(C1=N[C@@H]([2H])CN1)CO2] | (4S)-2-(2,3-dihydrobenzo[b][1,4]dioxin-2- yl-7-d)-4,5-dihydro-1H-imidazole-4-d |
| 10 | 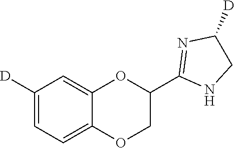 [SMILES: [2H]c1ccc2c(c1)OC(C1=N[C@H]([2H])CN1)CO2] | (4R)-2-(2,3-dihydrobenzo[b][1,4]dioxin-2- yl-7-d)-4,5-dihydro-1H-imidazole-4-d |
| 11 | 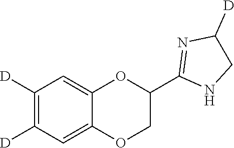 [SMILES: [2H]c1cc2c(cc1[2H])OC(C1=NC([2H])CN1)CO2] | 2-(2,3-dihydrobenzo[b][1,4]dioxin-2-yl-6,7- d2)-4,5-dihydro-1H-imidazole-4-d |
| 12 | 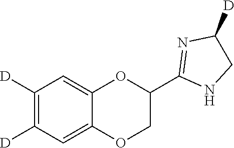 [SMILES: [2H]c1cc2c(cc1[2H])OC(C1=N[C@@H]([2H])CN1)CO2] | (4S)-2-(2,3-dihydrobenzo[b][1,4]dioxin-2- yl-6,7-d2)-4,5-dihydro-1H-imidazole-4-d |
| 13 | 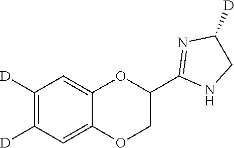 [SMILES: [2H]c1cc2c(cc1[2H])OC(C1=N[C@H]([2H])CN1)CO2] | (4R)-2-(2,3-dihydrobenzo[b][1,4]dioxin-2- yl-6,7-d2)-4,5-dihydro-1H-imidazole-4-d |
| 14 | 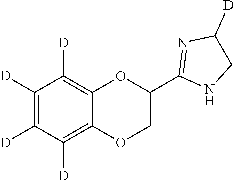 [SMILES: [2H]c1c([2H])c([2H])c2c(c1[2H])OCC(C1=NC([2H])CN1)O2] | 2-(2,3-dihydrobenzo[b][1,4]dioxin-2-yl- 5,6,7,8-d4)-4,5-dihydro-1H-imidazole-4-d |
| 15 | 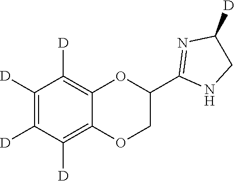 [SMILES: [2H]c1c([2H])c([2H])c2c(c1[2H])OCC(C1=N[C@@H]([2H])CN1)O2] | (4S)-2-(2,3-dihydrobenzo[b][1,4]dioxin-2- yl-5,6,7,8-d4)-4,5-dihydro-1H-imidazole-4-d |
| 16 | 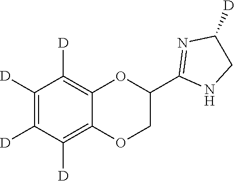 [SMILES: [2H]c1c([2H])c([2H])c2c(c1[2H])OCC(C1=N[C@H]([2H])CN1)O2] | (4R)-2-(2,3-dihydrobenzo[b][1,4]dioxin-2- yl-5,6,7,8-d4)-4,5-dihydro-1H-imidazole-4-d |
| 17 | 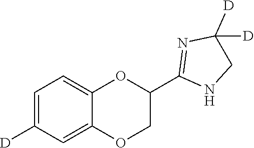 [SMILES: [2H]c1ccc2c(c1)OCC(C1=NC([2H])([2H])CN1)O2] | 2-(2,3-dihydrobenzo[b][1,4]dioxin-2-yl-6- d)-4,5-dihydro-1H-imidazole-4,4-d₂ |
| 18 | 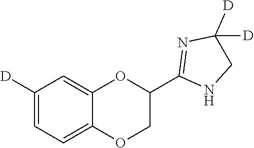 [SMILES: [2H]c1ccc2c(c1)OC(C1=NC([2H])([2H])CN1)CO2] | 2-(2,3-dihydrobenzo[b][1,4]dioxin-2-y1-7- d)-4,5-dihydro-1H-imidazole-4,4-d₂ |
| 19 | 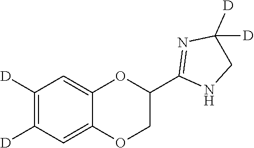 [SMILES: [2H]c1cc2c(cc1[2H])OC(C1=NC([2H])([2H])CN1)CO2] | 2-(2,3-dihydrobenzo[b][1,4]dioxin-2-yl-6,7- d2)-4,5-dihydro-1H-imidazole-4,4-d₂ |
| 20 | 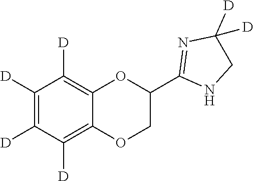 [SMILES: [2H]c1c([2H])c([2H])c2c(c1[2H])OCC(C1=NC([2H])([2H])CN1)O2] | 2-(2,3-dihydrobenzo[b][1,4]dioxin-2-yl- 5,6,7,8-d4)-4,5-dihydro-1H-imidazole-4,4-d₂ |
| 21 | 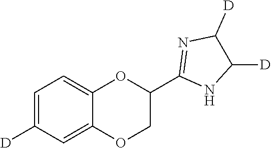 [SMILES: [2H]c1ccc2c(c1)OCC(C1=NC([2H])C([2H])N1)O2] | 2-(2,3-dihydrobenzo[b][1,4]dioxin-2-yl-6- d)-4,5-dihydro-1H-imidazole-4,5-d₂ |
| 22 | 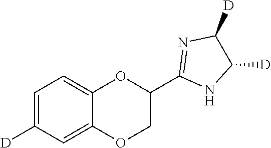 [SMILES: [2H]c1ccc2c(c1)OCC(C1=N[C@@H]([2H])[C@H]([2H])N1)O2] | (4S,5S)-2-(2,3-dihydrobenzo[b][1,4]dioxin- 2-yl-6-d)-4,5-dihydro-1H-imidazole-4,5-d₂ |
| 23 | 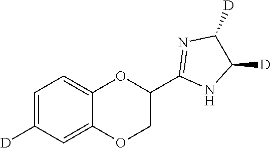 [SMILES: [2H]c1ccc2c(c1)OCC(C1=N[C@H]([2H])[C@@H]([2H])N1)O2] | (4R,5R)-2-(2,3-dihydrobenzo[b][1,4]dioxin- 2-yl-6-d)-4,5-dihydro-1H-imidazole-4,5-d₂ |
| 24 | 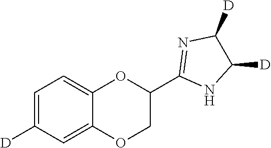 [SMILES: [2H]c1ccc2c(c1)OCC(C1=N[C@@H]([2H])[C@@H]([2H])N1)O2] | (4S,5R)-2-(2,3-dihydrobenzo[b][1,4]dioxin- 2-yl-6-d)-4,5-dihydro-1H-imidazole-4,5-d₂ |
| 25 | 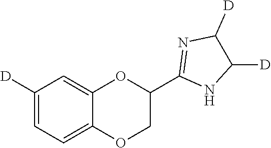 [SMILES: [2H]c1ccc2c(c1)OC(C1=NC([2H])C([2H])N1)CO2] | 2-(2,3-dihydrobenzo[b][1,4]dioxin-2-yl-7- d)-4,5-dihydro-1H-imidazole-4,5-d₂ |
| 26 | 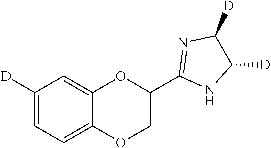 [SMILES: [2H]c1ccc2c(c1)OC(C1=N[C@@H]([2H])[C@H]([2H])N1)CO2] | (4S,5S)-2-(2,3-dihydrobenzo[b][1,4]dioxin- 2-yl-7-d)-4,5-dihydro-1H-imidazole-4,5-d₂ |
| 27 | 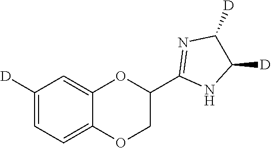 [SMILES: [2H]c1ccc2c(c1)OC(C1=N[C@H]([2H])[C@@H]([2H])N1)CO2] | (4R,5R)-2-(2,3-dihydrobenzo[b][1,4]dioxin- 2-yl-7-d)-4,5-dihydro-1H-imidazole-4,5-d₂ |
| 28 | 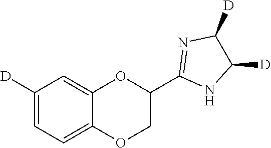 [SMILES: [2H]c1ccc2c(c1)OC(C1=N[C@@H]([2H])[C@@H]([2H])N1)CO2] | (4S,5R)-2-(2,3-dihydrobenzo[b][1,4]dioxin- 2-yl-7-d)-4,5-dihydro-1H-imidazole-4,5-d₂ |
| 29 | 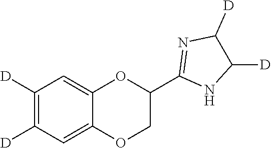 [SMILES: [2H]c1cc2c(cc1[2H])OC(C1=NC([2H])C([2H])N1)CO2] | 2-(2,3-dihydrobenzo[b][1,4]dioxin-2-yl-6,7- d2)-4,5-dihydro-1H-imidazole-4,5-d₂ |
| 30 | 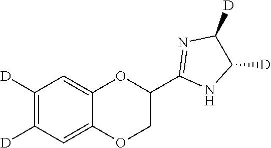 [SMILES: [2H]c1cc2c(cc1[2H])OC(C1=N[C@@H]([2H])[C@H]([2H])N1)CO2] | (4S,5S)-2-(2,3-dihydrobenzo[b][1,4]dioxin- 2-yl-6,7-d2)-4,5-dihydro-1H-imidazole-4,5- d₂ |
| 31 | 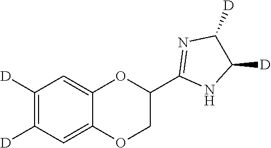 [SMILES: [2H]c1cc2c(cc1[2H])OC(C1=N[C@H]([2H])[C@@H]([2H])N1)CO2] | (4R,5R)-2-(2,3-dihydrobenzo[b][1,4]dioxin- 2-yl-6,7-d2)-4,5-dihydro-1H-imidazole-4,5- d₂ |
| 32 | 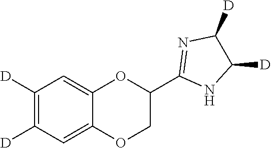 [SMILES: [2H]c1cc2c(cc1[2H])OC(C1=N[C@@H]([2H])[C@@H]([2H])N1)CO2] | (4S,5R)-2-(2,3-dihydrobenzo[b][1,4]dioxin- 2-yl-6,7-d2)-4,5-dihydro-1H-imidazole-4,5- d₂ |
| 33 | 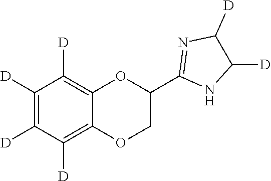 [SMILES: [2H]c1c([2H])c([2H])c2c(c1[2H])OCC(C1=NC([2H])C([2H])N1)O2] | 2-(2,3-dihydrobenzo[b][1,4]dioxin-2-yl- 5,6,7,8-d4)-4,5-dihydro-1H-imidazole-4,5-d₂ |
| 34 | 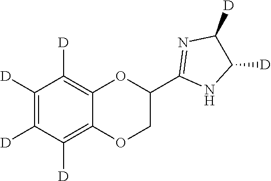 [SMILES: [2H]c1c([2H])c([2H])c2c(c1[2H])OCC(C1=N[C@@H]([2H])[C@H]([2H])N1)O2] | (4S,5S)-2-(2,3-dihydrobenzo[b][1,4]dioxin- 2-yl-5,6,7,8-d4)-4,5-dihydro-1H-imidazole- 4,5-d₂ |
| 35 | 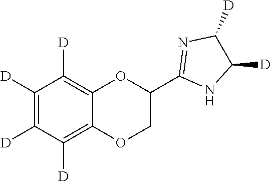 [SMILES: [2H]c1c([2H])c([2H])c2c(c1[2H])OCC(C1=N[C@H]([2H])[C@@H]([2H])N1)O2] | (4R,5R)-2-(2,3-dihydrobenzo[b][1,4]dioxin- 2-yl-5,6,7,8-d4)-4,5-dihydro-1H-imidazole- 4,5-d₂ |
| 36 | 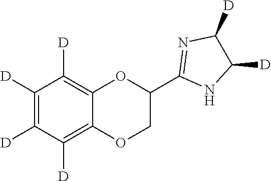 [SMILES: [2H]c1c([2H])c([2H])c2c(c1[2H])OCC(C1=N[C@@H]([2H])[C@@H]([2H])N1)O2] | (4S,5R)-2-(2,3-dihydrobenzo[b][1,4]dioxin- 2-yl-5,6,7,8-d4)-4,5-dihydro-1H-imidazole- 4,5-d₂ |
| 37 | 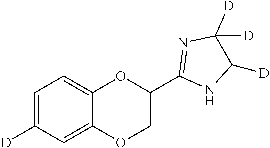 [SMILES: [2H]c1ccc2c(c1)OCC(C1=NC([2H])([2H])C([2H])N1)O2] | 2-(2,3-dihydrobenzo[b][1,4]dioxin-2-yl-6- d)-4,5-dihydro-1H-imidazole-4,4,5-d₃ |
| 38 | 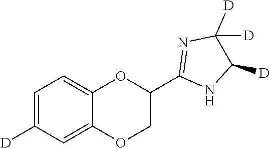 [SMILES: [2H]c1ccc2c(c1)OCC(C1=NC([2H])([2H])[C@@H]([2H])N1)O2] | (5R)-2-(2,3-dihydrobenzo[b][1,4]dioxin-2- yl-6-d)-4,5-dihydro-1H-imidazole-4,4,5-d₃ |
| 39 | 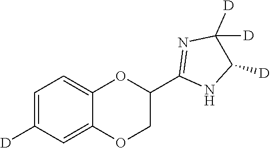 [SMILES: [2H]c1ccc2c(c1)OCC(C1=NC([2H])([2H])[C@H]([2H])N1)O2] | (5S)-2-(2,3-dihydrobenzo[b][1,4]dioxin-2- yl-6-d)-4,5-dihydro-1H-imidazole-4,4,5-d₃ |
| 40 | 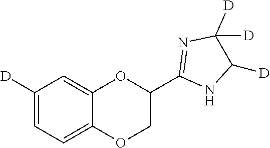 [SMILES: [2H]c1ccc2c(c1)OC(C1=NC([2H])([2H])C([2H])N1)CO2] | 2-(2,3-dihydrobenzo[b][1,4]dioxin-2-y1-7- d)-4,5-dihydro-1H-imidazole-4,4,5-d₃ |
| 41 | 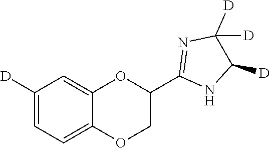 [SMILES: [2H]c1ccc2c(c1)OC(C1=NC([2H])([2H])[C@@H]([2H])N1)CO2] | (5R)-2-(2,3-dihydrobenzo[b][1,4]dioxin-2- yl-7-d)-4,5-dihydro-1H-imidazole-4,4,5-d₃ |
| 42 | 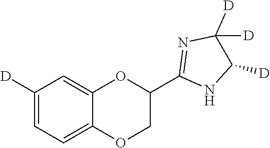 [SMILES: [2H]c1ccc2c(c1)OC(C1=NC([2H])([2H])[C@H]([2H])N1)CO2] | (5S)-2-(2,3-dihydrobenzo[b][1,4]dioxin-2- yl-7-d)-4,5-dihydro-1H-imidazole-4,4,5-d₃ |
| 43 | 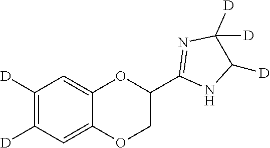 [SMILES: [2H]c1cc2c(cc1[2H])OC(C1=NC([2H])([2H])C([2H])N1)CO2] | 2-(2,3-dihydrobenzo[b][1,4]dioxin-2-yl-6,7- d2)-4,5-dihydro-1H-imidazole-4,4,5-d₃ |
| 44 | 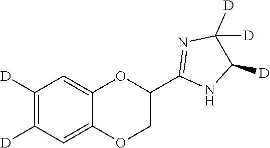 [SMILES: [2H]c1cc2c(cc1[2H])OC(C1=NC([2H])([2H])[C@@H]([2H])N1)CO2] | (5R)-2-(2,3-dihydrobenzo[b][1,4]dioxin-2- yl-6,7-d2)-4,5-dihydro-1H-imidazole-4,4,5- d₃ |
| 45 | 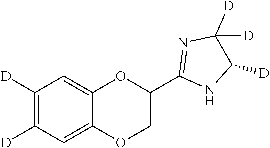 [SMILES: [2H]c1cc2c(cc1[2H])OC(C1=NC([2H])([2H])[C@H]([2H])N1)CO2] | (5S)-2-(2,3-dihydrobenzo[b][1,4]dioxin-2- yl-6,7-d2)-4,5-dihydro-1H-imidazole-4,4,5- d₃ |
| 46 | 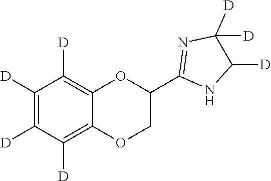 [SMILES: [2H]c1c([2H])c([2H])c2c(c1[2H])OCC(C1=NC([2H])([2H])C([2H])N1)O2] | 2-(2,3-dihydrobenzo[b][1,4]dioxin-2-yl- 5,6,7,8-d4)-4,5-dihydro-1H-imidazole-4,4,5- d₃ |
| 47 | 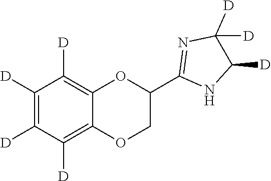 [SMILES: [2H]c1c([2H])c([2H])c2c(c1[2H])OCC(C1=NC([2H])([2H])[C@@H]([2H])N1)O2] | (5R)-2-(2,3-dihydrobenzo[b][1,4]dioxin-2- yl-5,6,7,8-d4)-4,5-dihydro-1H-imidazole- 4,4,5-d₃ |
| 48 | 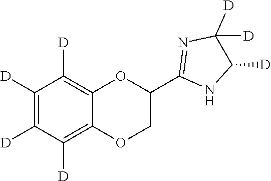 [SMILES: [2H]c1c([2H])c([2H])c2c(c1[2H])OCC(C1=NC([2H])([2H])[C@H]([2H])N1)O2] | (5S)-2-(2,3-dihydrobenzo[b][1,4]dioxin-2- yl-5,6,7,8-d4)-4,5-dihydro-1H-imidazole- 4,4,5-d₃ |
| 49 | 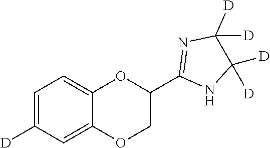 [SMILES: [2H]c1ccc2c(c1)OCC(C1=NC([2H])([2H])C([2H])([2H])N1)O2] | 2-(2,3-dihydrobenzo[b][1,4]dioxin-2-yl-6- d)-4,5-dihydro-1H-imidazole-4,4,5,5-d₄ |
| 49-R | 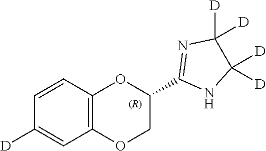 | (R)-2-(2,3-dihydrobenzo[b][1,4]dioxin-2-yl- 6-d)-4,5-dihydro-1H-imidazole-4,4,5,5-d4 |
| 49-S | 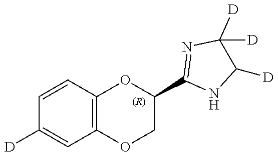 | (S)-2-(2,3-dihydrobenzo[b][1,4]dioxin-2-yl- 6-d)-4,5-dihydro-1H-imidazole-4,4,5,5-d4 |
| 50 | 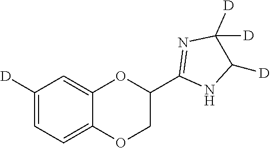 [SMILES: [2H]c1ccc2c(c1)OC(C1=NC([2H])([2H])C([2H])N1)CO2] | 2-(2,3-dihydrobenzo[b][1,4]dioxin-2-y1-7- d)-4,5-dihydro-1H-imidazole-4,4,5,5-d₄ |
| 50-R | 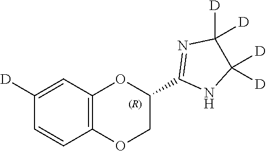 | (R)-2-(2,3-dihydrobenzo[b][1,4]dioxin-2-yl- 7-d)-4,5-dihydro-1H-imidazole-4,4,5,5-d4 |
| 50-S | 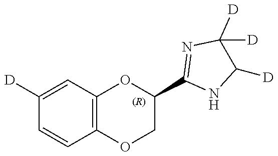 | (S)-2-(2,3-dihydrobenzo[b][1,4]dioxin-2-yl- 7-d)-4,5-dihydro-1H-imidazole-4,4,5,5-d4 |
| 51 | 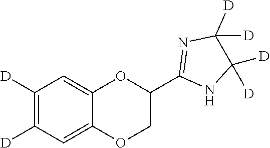 [SMILES: [2H]c1cc2c(cc1[2H])OC(C1=NC([2H])([2H])C([2H])([2H])N1)CO2] | 2-(2,3-dihydrobenzo[b][1,4]dioxin-2-y1-6,7- d₂)-4,5-dihydro-1H-imidazole-4,4,5,5-d₄ |
| 51-R | 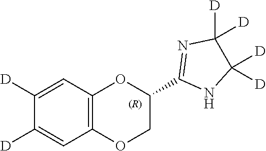 | (R)-2-(2,3-dihydrobenzo[b][1,4]dioxin-2-yl- 6,7-d2)-4,5-dihydro-1H-imidazole-4,4,5,5- d4 |
| 51-S | 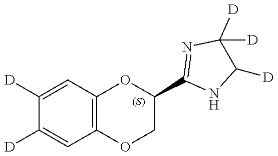 | (S)-2-(2,3-dihydrobenzo[b][1,4]dioxin-2-yl- 6,7-d2)-4,5-dihydro-1H-imidazole-4,4,5,5- d4 |
| 52 | 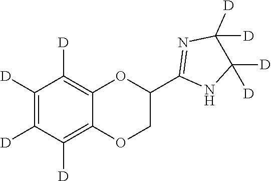 [SMILES: [2H]c1c([2H])c([2H])c2c(c1[2H])OCC(C1=NC([2H])([2H])C([2H])([2H])N1)O2] | 2-(2,3-dihydrobenzo[b][1,4]dioxin-2-yl- 5,6,7,8-d4)-4,5-dihydro-1H-imidazole- 4,4,5,5-d₄ |
| 52-R | 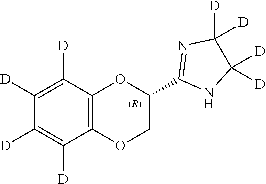 | (R)-2-(2,3-dihydrobenzo[b][1,4]dioxin-2-yl- 5,6,7,8-d4)-4,5-dihydro-1H-imidazole- 4,4,5,5-d4 |
| 52-S | 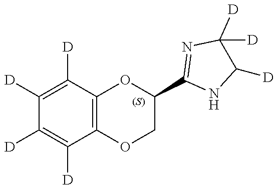 | (S)-2-(2,3-dihydrobenzo[b][1,4]dioxin-2-yl- 5,6,7,8-d4)-4,5-dihydro-1H-imidazole- 4,4,5,5-d4 |
| 53 | 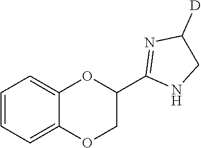 [SMILES: [2H]C1CNC(C2COc3ccccc3O2)=N1] | 2-(2,3-dihydrobenzo[b][1,4]dioxin-2-yl)- 4,5-dihydro-1H-imidazole-4-d |
| 54 | 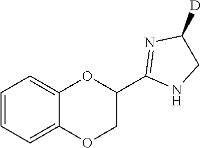 [SMILES: [2H][C@H]1CNC(C2COc3ccccc3O2)=N1] | (4S)-2-(2,3-dihydrobenzo[b][1,4]dioxin-2- yl)-4,5-dihydro-1H-imidazole-4-d |
| 55 | 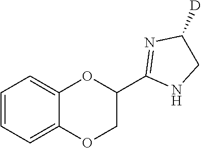 [SMILES: [2H][C@@H]1CNC(C2COc3ccccc3O2)=N1] | (4R)-2-(2,3-dihydrobenzo[b][1,4]dioxin-2- yl)-4,5-dihydro-1H-imidazole-4-d |
| 56 | 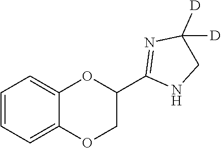 [SMILES: [2H]C1([2H])CNC(C2COc3ccccc3O2)=N1] | 2-(2,3-dihydrobenzo[b][1,4]dioxin-2-yl)- 4,5-dihydro-1H-imidazole-4,4-d₂ |
| 57 | 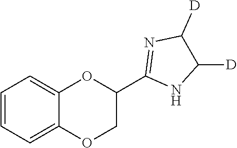 [SMILES: [2H]C1N=C(C2COc3ccccc3O2)NC1[2H]] | 2-(2,3-dihydrobenzo[b][1,4]dioxin-2-yl)- 4,5-dihydro-1H-imidazole-4,5-d₂ |
| 58 | 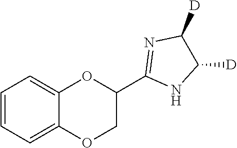 [SMILES: [2H][C@@H]1N=C(C2COc3ccccc3O2)N[C@H]1[2H]] | (4S,5S)-2-(2,3-dihydrobenzo[b][1,4]dioxin- 2-yl)-4,5-dihydro-1H-imidazole-4,5-d₂ |
| 59 | 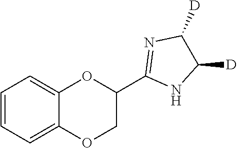 [SMILES: [2H][C@H]1N=C(C2COc3ccccc3O2)N[C@@H]1[2H]] | (4R,5R)-2-(2,3-dihydrobenzo[b][1,4]dioxin- 2-yl)-4,5-dihydro-1H-imidazole-4,5-d₂ |
| 60 | 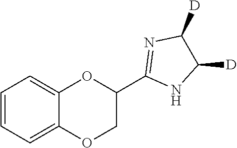 [SMILES: [2H][C@@H]1N=C(C2COc3ccccc3O2)N[C@@H]1[2H]] | (4S,5R)-2-(2,3-dihydrobenzo[b][1,4]dioxin- 2-yl)-4,5-dihydro-1H-imidazole-4,5-d₂ |
| 61 | 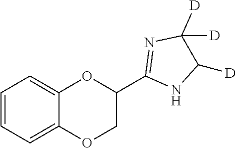 [SMILES: [2H]C1NC(C2COc3ccccc3O2)=NC1([2H])[2H]] | 2-(2,3-dihydrobenzo[b][1,4]dioxin-2-yl)- 4,5-dihydro-1H-imidazole-4,4,5-d₃ |
| 62 | 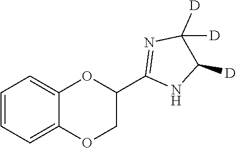 [SMILES: [2H][C@H]1NC(C2COc3ccccc3O2)=NC1([2H])[2H]] | (5R)-2-(2,3-dihydrobenzo[b][1,4]dioxin-2- yl)-4,5-dihydro-1H-imidazole-4,4,5-d₃ |
| 63 | 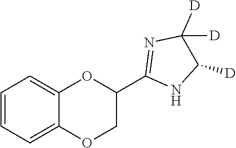 [SMILES: [2H][C@@H]1NC(C2COc3ccccc3O2)=NC1([2H])[2H]] | (5S)-2-(2,3-dihydrobenzo[b][1,4]dioxin-2- yl)-4,5-dihydro-1H-imidazole-4,4,5-d₃ |
| 64 | 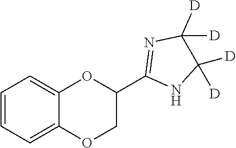 [SMILES: [2H]C1([2H])N=C(C2COc3ccccc3O2)NC1([2H])[2H]] | 2-(2,3-dihydrobenzo[b][1,4]dioxin-2-yl)- 4,5-dihydro-1H-imidazole-4,4,5,5-d₄ |
| 64-R | 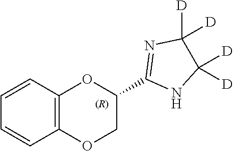 | (R)-2-(2,3-dihydrobenzo[b][1,4]dioxin-2- yl)-4,5-dihydro-1H-imidazole-4,4,5,5-d4 |
| 64-S | 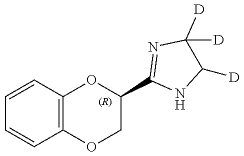 | (S)-2-(2,3-dihydrobenzo[b][1,4]dioxin-2-yl)- 4,5-dihydro-1H-imidazole-4,4,5,5-d4 |

Some of the foregoing compounds can comprise one or more asymmetric centers, and thus can exist in various isomeric forms, e.g., stereoisomers and/or diastereomers. Accordingly, compounds of the application may be in the form of an individual enantiomer, diastereomer or geometric isomer, or may be in the form of a mixture of stereoisomers. In some embodiments, the compounds of the application are enantiopure compounds. In another embodiment, mixtures of stereoisomers or diastereomers are provided.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four non-identical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center, e.g., carbon. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose. Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine. The compounds of this application may also be represented in multiple tautomeric forms, in such instances, the application expressly includes all tautomeric forms of the compounds described herein (e.g., alkylation of a ring system may result in alkylation at multiple sites, the application expressly includes all such reaction products).

In the present application, the structural formula of the compound represents a certain isomer for convenience in some cases, but the present application includes all isomers, such as geometrical isomers, optical isomers based on an asymmetrical carbon, stereoisomers, tautomers, and the like.

For example, the tautomer represented by Formula (I) below, can exist in both forms:

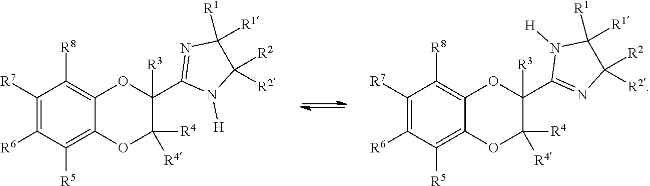

In some embodiments, both of the above tautomers are encompassed when one or the other form is depicted.

In one aspect, the isotopically labeled compound of any of the formulae herein may have one or more additional isotope atoms which may or may not be radioactive (e.g., ³H, ¹⁴C, ¹³C, ¹⁸F, ³⁵S, ³²P, ¹²⁵I, and ¹³¹I) introduced into the compound. Such compounds are useful for drug metabolism studies and diagnostics, as well as therapeutic applications.

Potency can also be determined by IC₅₀ value. A compound with a lower IC₅₀ value, as determined under substantially similar conditions, is more potent relative to a compound with a higher IC₅₀ value.

In some embodiments, the compounds of the present application are useful as therapeutic agents, and thus may be useful in the treatment of a disease caused by, or associated with, a neurological condition

The compounds of the application are defined herein by their chemical structures and/or chemical names. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

In another aspect, the application provides a method of synthesizing a compound disclosed herein. The synthesis of the compounds of the application can be found herein and in the Examples below. Other embodiments are a method of making a compound of any of the formulae herein using any one, or combination of, reactions delineated herein. The method can include the use of one or more intermediates or chemical reagents delineated herein.

The application also provides for a pharmaceutical composition comprising a therapeutically effective amount of a compound of the application, or a pharmaceutically acceptable salt, solvate, hydrate, tautomer, or stereoisomer thereof, and a pharmaceutically acceptable carrier.

Another aspect of the present application relates to a kit comprising a compound of the application or a pharmaceutically acceptable salt, solvate, hydrate, tautomer, or stereoisomer thereof, or a pharmaceutical composition of the application. In another aspect, the application provides a kit comprising a compound capable of treating a neurological condition selected from one or more compounds disclosed herein, or a pharmaceutically acceptable salt, solvate, hydrate, tautomer, or stereoisomer thereof, optionally in combination with a second agent and instructions for use.

In some embodiments, the compounds of the present application form less of one or more metabolites compared to the non-deuterated analog. In some embodiments, the compounds of the present application form about 10% less, about 20% less, about 30% less, about 40% less, about 50% less, about 60% less, about 70% less, about 80% less, about 90% less, or about 100% less of one or more metabolites compared to the non-deuterated analog. In some embodiments, the compounds of the present application form about 10% less, about 20% less, about 30% less, about 40% less, about 50% less, about 60% less, about 70% less, about 80% less, about 90% less, or about 100% less of the 6-OH metabolite compared to the non-deuterated analog. In some embodiments, the compounds of the present application form about 10% less, about 20% less, about 30% less, about 40% less, about 50% less, about 60% less, about 70% less, 80% less, 90% less, or 100% less of the 7-OH metabolite compared to the non-deuterated analog.

In some embodiments, the compounds of the present application have an increased bioavailability compared to the non-deuterated analog. In some embodiments, the compounds of the present application have an increased bioavailability about 10%, about 20%, about 30%, about 40%, about 50%, about 60%, about 70%, about 80%, about 90%, or about 100% higher compared to the non-deuterated analog.

In some embodiments, the compounds of the present application maintain in systemic circulation longer compared to the non-deuterated analog. In some embodiments, the compounds of the present application stay in systemic circulation for about 10%, about 20%, about 30%, about 40%, about 50%, about 60%, about 70%, about 80%, about 90%, or about 100% longer compared to the non-deuterated analog.

### Methods of Synthesizing the Compounds

Compounds of the present application can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the present application. The processes generally provide the desired final compound at or near the end of the overall process, although it may be desirable in certain instances to further convert the compound to a pharmaceutically acceptable salt, ester, or prodrug thereof. Suitable synthetic routes are depicted in the schemes below.

Those skilled in the art will recognize if a stereocenter exists in the compounds disclosed herein. Accordingly, the present application includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds of the present application can be prepared in a number of ways well known to those skilled in the art of organic synthesis. By way of example, compounds of the present application can be synthesized using the methods described below, together with synthetic methods known in the art of synthetic organic chemistry, or variations thereon as appreciated by those skilled in the art. Preferred methods include but are not limited to those methods described below.

Compounds of the present application can be synthesized by following the steps outlined in the following Schemes, which comprise different sequences of assembling intermediates. Starting materials are either commercially available or made by known procedures in the reported literature or as illustrated in the schemes below.

A compound of the application can be prepared as a pharmaceutically acceptable acid addition salt by reacting the free base form of the compound with a pharmaceutically acceptable inorganic or organic acid. Alternatively, a pharmaceutically acceptable base addition salt of a compound of the application can be prepared by reacting the free acid form of the compound with a pharmaceutically acceptable inorganic or organic base. The pharmaceutically acceptable salt may include various counterions, e.g., counterions of the inorganic or organic acid, counterions of the inorganic or organic base, or counterions afforded by counterion exchange.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Alternatively, the salt forms of the compounds of the application can be prepared using salts of the starting materials or intermediates. The free acid or free base forms of the compounds of the application can be prepared from the corresponding base addition salt or acid addition salt from, respectively. For example, a compound of the application in an acid addition salt form can be converted to the corresponding free base by treating with a suitable base (e.g., ammonium hydroxide solution, sodium hydroxide, and the like). A compound of the application in a base addition salt form can be converted to the corresponding free acid by treating with a suitable acid (e.g., hydrochloric acid, etc.).

Compounds of the present application that contain nitrogens can be converted to N-oxides by treatment with an oxidizing agent (e.g., 3-chloroperoxybenzoic acid (m-CPBA) and/or hydrogen peroxides) to afford other compounds of the present application. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present application can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

Prodrugs of the compounds of the application can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the application with a suitable carbamylating agent (e.g., 1,1-acyloxyalkylcarbanochloridate, para-nitrophenyl carbonate, or the like). Specifically, the central N-acetic acid moeity, and other analogous carboxylic acid groups, of the compounds of the present invention can be modified through techniques known in the art to produce effective prodrugs of the present invention.

Protected derivatives of the compounds of the application can be made by means known to those of ordinary skill in the art. A detailed description of techniques applicable to the creation of protecting groups and their removal can be found in T. W. Greene, “Protecting Groups in Organic Chemistry”, 3rd edition, John Wiley and Sons, Inc., 1999.

Compounds of the present application can be conveniently prepared, or formed during the process of the application, as solvates (e.g., hydrates). Hydrates of compounds of the present application can be conveniently prepared by recrystallization from an aqueous/organic solvent mixture, using organic solvents such as dioxin, tetrahydrofuran or methanol.

Optical isomers may be prepared from their respective optically active precursors by the procedures described herein, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981).

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired bridged macrocyclic products of the present application. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this application may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

### Biological Assays

Biological activities of the compounds of the present application can be measured by various biochemical or cellular assays known to one of ordinary skill in the art. Non-limiting examples of methods for determining biological activity are discussed herein.

### Pharmacokinetic Profile for the Deuterated Compounds

The compounds of the present application can be measured for stability. Specifically, stability in microsomes using the protocol described herein and/or in WO 2020/132461, which is incorporated by reference herein in its entirety, may be used to assess the compounds of the present application.

### Evaluation of Metabolic Stability in Human Liver Microsomes

Pharmacokinetics of the compounds of the present application may be determined following a single intravenous or oral administration in one or more test subjects, including mice, rats, dogs, etc., A pharmacokinetic study can be performed in three male Sprague-Dawley (SD) rats following intravenous (IV) and oral (PO) administration of the compounds of the present disclosure in comparison with non-deuterated compounds. Various doses may be used according to the route of administration in the test subjects, for example at 1 mg/kg (IV) and 10 (PO) mg/kg. Test compounds can be measured in plasma and the values are determined according to known methods in the art.

### Pharmaceutical Compositions

In another aspect, a pharmaceutical composition is provided. The pharmaceutical composition comprises a therapeutically effective amount of a compound of the application, or a pharmaceutically acceptable salt, solvate, hydrate, tautomer, or stereoisomer thereof, and a pharmaceutically acceptable carrier.

Compounds of the application may be administered as pharmaceutical compositions by any conventional route, in particular enterally, e.g., orally, e.g., in the form of tablets or capsules, or parenterally, e.g., in the form of injectable solutions or suspensions, or topically, e.g., in the form of lotions, gels, ointments or creams, or in a nasal or suppository form.

Pharmaceutical compositions including a compound of the present application in free form or in a pharmaceutically acceptable salt form in association with at least one pharmaceutically acceptable carrier or diluent may be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present application with a carrier. A carrier may include absorbable pharmacologically acceptable solvents to assist passage through the skin of the host. For example, transdermal devices may be in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The pharmaceutical compositions of the present application comprise a therapeutically effective amount of a compound of the present application formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which may serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxy propylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this application may be administered to humans and other animals orally, rectally, parenterally, intracisternally, intravaginally, intraperitoneally, topically (as by powders, ointments, or drops), buccally, or as an oral or nasal spray.

Liquid dosage forms for oral administration may include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous, or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds may also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

In some embodiments, a pharmaceutical composition of the present disclosure is a solid dosage form that is formulated for immediate release (IR). In some embodiments, a pharmaceutical composition of the present disclosure is a solid dosage form that is formulated for extended release (ER).

As used herein, the term “immediate release formulation” refers compositions comprising a compound of the application that lack a release modifying structural element such as a matrix or coating.

As used herein the term “extended release” refers to a mode of releasing an active ingredient from a pharmaceutical composition in which the timing and/or rate of release of the active ingredient is delayed or slowed. A pharmaceutical composition that is formulated for extended release is a pharmaceutical composition that comprises one or more structural features that have the effect of preventing active ingredient from being released immediately upon administration to a subject. For example, in pharmaceutical composition that is formulated for extended release, the active ingredient may be embedded within a matrix core that degrades slowly over time. Alternatively or additionally, the pharmaceutical composition may comprise a polymeric coating that loses structural integrity over time, allowing active ingredient previously enclosed by the coating to be released.

In some embodiments, a pharmaceutical composition of the present comprises a racemic mixture of deuterated analogues of the (R) and (S) enantiomers of idazoxan. In some embodiments, a pharmaceutical composition of the present comprises a mixture of deuterated analogues of the (R) and (S) enantiomers of idazoxan that is enriched for the (R) enantiomer. In some embodiments, a pharmaceutical composition of the present comprises deuterated analogues of the (R) enantiomer of idazoxan. In some embodiments, a pharmaceutical composition of the present comprises a mixture of deuterated analogues of the (R) and(S) enantiomers of idazoxan that is enriched for the(S) enantiomer. In some embodiments, a pharmaceutical composition of the present comprises deuterated analogues of the(S) enantiomer of idazoxan.

Without wishing to be bound by theory, it is believed that administration of a deuterated analogue of (R)-idazoxan, the inactive enantiomer of idazoxan, to a subject results in the formation of a depot of a deuterated analogue of (R)-idazoxan in the subject. It is believed that, over time, the depot of a deuterated analogue of (R)-idazoxan converts to(S)-idazoxan. It is believed that the conversion of (R) to (S) over time results in persistent exposure to a deuterated analogue of(S) idazoxan, e.g., at concentrations within the therapeutic window of idazoxan. This means that formulations of a deuterated analogue of (R)-idazoxan may possess an improved ability to maintain the deuterated(S)-idazoxan AUC in the optimal range which exhibits the desirable therapeutic effects over formulations of a deuterated analogue of (S)-idazoxan and a deuterated analogue of racemic-idazoxan.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this application, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. 7. Long-acting pharmaceutical compositions may be administered every 3 to 4 days, every week, or once every two weeks depending on half-life and clearance rate of the particular formulation.

The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this application include powders, sprays, ointments, pastes, creams, lotions, gels, solutions, patches and inhalants. In some embodiments, the active compound is mixed under sterile conditions with a pharmaceutically acceptable carrier, and with any preservatives, buffers or propellants that are required.

The pharmaceutical compositions containing active compounds of the present application may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Techniques for formulation and administration of the disclosed compounds of the application can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

### Methods of Use

The compounds of the present disclosure are also used in the manufacture of a medicament, in particular in the manufacture of a medicament intended for the preventive and/or curative treatment of disorders of the central nervous system. In some embodiments, the medicament in the form of a tablet is intended for the treatment of depression, Parkinson's disease and/or severe psychotic disorders, such as schizophrenia and schizoaffective disorders.

In some embodiments, the present application provides a method of treating or preventing any of the diseases, disorders, and conditions described herein, wherein the subject is a human. In some embodiments, the application provides a method of treating. In some embodiments, the application provides a method of preventing.

In some embodiments, the brain disorder is selected from Alzheimer's disease, ataxia, Huntington's disease, Parkinson's disease, Creutzfeldt-Jakob disease, amyotrophic later sclerosis, motor neuron disease, multiple system atrophy, or progressive supranuclear palsy, vascular dementia, Lewy body dementia, frontotemporal dementia, frontal lobe dementia, or mixed dementia, migraine, tension headache, cluster headache, depression, treatment resistant depression, postpartum depression, psychotic depression, seasonal affective disorder, major depressive disorder, persistent depressive disorder, bipolar depression, premenstrual dysphoric disorder, addiction, substance use disorder, anxiety, post-traumatic stress disorder, suicidal ideation, major depressive disorder, psychosis, schizophrenia, stroke, traumatic brain injury, generalized anxiety disorder, panic disorder, social anxiety disorder, one or more phobia-related disorders, separation anxiety disorder, substance use disorder, or a schizoaffective disorder, or a combination thereof.

In some embodiments, the brain disorder is a schizoaffective disorder. In some embodiments, the brain disorder is schizophrenia.

In some embodiments, the brain disorder is a schizoaffective disorder. In some embodiments, the brain disorder is a type of dementia.

In some embodiments, the brain disorder is a schizoaffective disorder. In some embodiments, the brain disorder is Alzheimer's disease. In some embodiments, the pharmaceutical composition is administered in combination with a second therapeutic agent.

In some embodiments, the pharmaceutical composition is administered in combination with an antipsychotic. In some embodiments, the pharmaceutical composition is administered in combination with an antagonist of the dopamine type 2 receptor (D2 antagonist).

In some embodiments, the pharmaceutical composition is administered for a period of one week, two weeks, three weeks, four weeks, five weeks, six weeks, seven weeks, eight weeks, nine weeks, or ten weeks. In some embodiments, the pharmaceutical composition is administered for a period of one month, two months, three months, four months, five months, six months, seven months, eight months, nine months, ten months, eleven months, or 12 months. In some embodiments, the pharmaceutical composition is administered for a period of one year, two years, three years, four years, five years, six years, seven years, eight years, nine years, ten months, eleven years, or 12 years. In some embodiments, the pharmaceutical composition is administered for the lifetime of the subject.

In accordance with the foregoing, the present application provides a method for preventing or treating any of the diseases or disorders described herein in a subject in need of such treatment, comprising administering to the subject a therapeutically effective amount of a compound of the application or an enantiomer, diastereomer, stereoisomer, or pharmaceutically acceptable salt thereof, or a pharmaceutical composition of the application. For any of the above uses, the required dosage will vary depending on the mode of administration, the particular condition to be treated and the effect desired.

Combination therapy may include the administration of the subject compounds in further combination with one or more other biologically active ingredients. For instance, the compounds of the application can be used in combination with other pharmaceutically active compounds, preferably compounds that are able to enhance the effect of the compounds of the application. The compounds of the application can be administered simultaneously (as a single preparation or separate preparation) or sequentially to the other drug therapy or treatment modality. In general, a combination therapy envisions administration of two or more drugs during a single cycle or course of therapy.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

“Treat”, “treating” and “treatment” refer to a method of alleviating or abating a disease and/or its attendant symptoms.

As used herein, “preventing” or “prevent” describes reducing or eliminating the onset of the symptoms or complications of the disease, condition or disorder.

The terms “disease(s)”, “disorder(s)”, and “condition(s)” are used interchangeably, unless the context clearly dictates otherwise.

The term “therapeutically effective amount” of a compound or pharmaceutical composition of the application, as used herein, means a sufficient amount of the compound or pharmaceutical composition so as to decrease the symptoms of a disorder in a subject. As is well understood in the medical arts a therapeutically effective amount of a compound or pharmaceutical composition of this application will be at a reasonable benefit/risk ratio applicable to any medical treatment. It will be understood, however, that the total daily usage of the compounds and compositions of the present application will be decided by the attending physician within the scope of sound medical judgment. The specific modulatory (e.g., inhibitory or stimulatory) dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable salt” refers to those salts of the compounds formed by the process of the present application which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in. J. Pharmaceutical Sciences, 66: 1-19 (1977). The salts can be prepared in situ during the final isolation and purification of the compounds of the application, or separately by reacting the free base or acid function with a suitable acid or base.

Examples of pharmaceutically acceptable salts include, but are not limited to, nontoxic acid addition salts: salts formed with inorganic acids such as hydrochloric acid, hydrobromic acid, phosphoric acid, sulfuric acid and perchloric acid, or with organic acids such as acetic acid, maleic acid, tartaric acid, citric acid, succinic acid or malonic acid. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, 7-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters of the compounds formed by the process of the present application which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein, refers to those prodrugs of the compounds formed by the process of the present application which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present application.

“Prodrug”, as used herein, means a compound which is convertible in vivo by metabolic means (e.g., by hydrolysis) to afford any compound delineated by the formulae of the instant application. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

This application also encompasses pharmaceutical compositions containing, and methods of treating disorders through administering, pharmaceutically acceptable prodrugs of compounds of the application. For example, compounds of the application having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the application. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1-15. Carbamate prodrugs of hydroxy and amino groups are also included, as are carbonate prodrugs, sulfonate esters and sulfate esters of hydroxy groups. Derivatization of hydroxy groups as (acyloxy) methyl and (acyloxy) ethyl ethers wherein the acyl group may be an alkyl ester, optionally substituted with groups including but not limited to ether, amine and carboxylic acid functionalities, or where the acyl group is an amino acid ester as described herein, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

Combinations of substituents and variables envisioned by this application are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

When any variable (e.g., R₁) occurs more than one time in any constituent or formula for a compound, its definition at each occurrence is independent of its definition at every other occurrence. Thus, for example, if a group is shown to be substituted with one or more R moieties, then R at each occurrence is selected independently from the definition of R. Also, combinations of substituents and/or variables are permissible, but only if such combinations result in stable compounds within a designated atom's normal valency.

In addition, some of the compounds of this application have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. When the compounds described herein contain olefinic double bonds or other centers of geometric asymmetry, and unless specified otherwise, it is intended that the compounds include both E and Z geometric isomers. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included in the present application.

Additionally, the compounds of the present application, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

### Methods of Treatment

In some embodiments, the pharmaceutical compositions of idazoxan disclosed herein may be used in the treatment of a disease or disorder. In some embodiments, the disease or disorder is a brain disorder. In some embodiments, the brain disorder is a neurodegenerative disorder such as Alzheimer's disease, ataxia, Huntington's disease, Parkinson's disease, Creutzfeldt-Jakob disease, amyotrophic later sclerosis, motor neuron disease, multiple system atrophy, or progressive supranuclear palsy. In some embodiments, the brain disorder is dementia such as vascular dementia, Lewy body dementia, frontotemporal dementia, frontal lobe dementia, or mixed dementia.

In some embodiments, the disease or disorder is diabetes mellitus.

In some embodiments, the brain disorder is migraine, tension headache, cluster headache.

In some embodiments, the brain disorder is a psychological disorder such as depression, treatment resistant depression, postpartum depression, psychotic depression, seasonal affective disorder, major depressive disorder, persistent depressive disorder, bipolar depression, premenstrual dysphoric disorder, addiction, substance use disorder, anxiety, post-traumatic stress disorder, suicidal ideation, major depressive disorder, psychosis, schizophrenia, stroke, traumatic brain injury, generalized anxiety disorder, panic disorder, social anxiety disorder, one or more phobia-related disorders, separation anxiety disorder, or substance use disorder. In some embodiments, the brain disorder is schizophrenia.

In some embodiments, the brain disorder is a schizoaffective disorder. In some embodiments, the schizoaffective disorder is bipolar type schizoaffective disorder. In some embodiments, the schizoaffective disorder is depressive type schizoaffective disorder. In some embodiments, the schizoaffective disorder is mixed type schizoaffective disorder.

In some embodiments, the disease or disorder is a musculoskeletal pain disorder including fibromyalgia, muscle pain, joint stiffness, osteoarthritis, rheumatoid arthritis, muscle cramps.

In some embodiments, the present disclosure provides a method of treating a disease of women's reproductive health including premenstrual dysphoric disorder (PMDD), premenstrual syndrome (PMS), post-partum depression, and menopause.

In some embodiments, the pharmaceutical compositions of idazoxan may be used to treat a combination of brain disorders.

In some embodiments, the pharmaceutical compositions of idazoxan have, anti-addictive properties, anti-psychotic properties, antidepressant properties, anxiolytic properties, or a combination thereof.

The compositions of idazoxan of the present disclosure can be used for increasing neuronal plasticity. The compounds of the present disclosure can also be used to treat any brain disorder. The compounds of the present disclosure can also be used for increasing at least one of translation, transcription or secretion of neurotrophic factors.

### Combination Therapy

In some embodiments, serotonin receptor modulators, such as modulators of serotonin receptor 2A (5-HT_{2A} modulators, e.g., 5-HT_{2A} agonists), are used to treat a brain disorder. The presently disclosed pharmaceutical compositions of idazoxan may be used in combination with a second therapeutic agent that also is a 5-HT_{2A} modulator. In such cases the second therapeutic agent can be an agonist or an antagonist. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methysergide, trazodone, cinitapride, cyproheptadine, brexpiprazole, cariprazine, agomelatine, setoperone, 1-(1-Naphthyl) piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, xanomeline, buspirone, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate (e.g., Prolixin Decanoate), an extended-release form of aripiprazole lauroxil (e.g., Aristada), an extended-release form of aripiprazole (e.g., Abilify Maintena), 3-(2-(4-(4-Fluorobenzoyl) piperazin-1-yl)ethyl)-5-methyl-5-phenylimidazolidine-2,4-dione, 3-(2-(4-Benzhydrylpiperazin-1-yl)ethyl)-5-methyl-5-phenylimidazolidine-2,4-dione, 3-(3-(4-(2-Fluorophenyl) piperazin-1-yl) propyl)-5-methyl-5-phenylimidazolidine-2,4-dione, 3-(3-(4-(3-Fluorophenyl) piperazin-1-yl) propyl)-5-methyl-5-phenylimidazolidine-2,4-dione, 3-(3-(4-(4-Fluorophenyl) piperazin-1-yl) propyl)-5-methyl-5-phenylimidazolidine-2,4-dione, 3-(3-(4-(4-Fluorobenzoyl) piperazin-1-yl) propyl)-5-methyl-5-phenylimidazolidine-2,4-dione, 3-(2-(4-(4-Fluorobenzoyl) piperazin-1-yl)ethyl)-8-phenyl-1,3-diazaspiro[4.5]decane-2,4-dione, 3-(2-(4-Benzhydrylpiperazin-1-yl)ethyl)-8-phenyl-1,3-diazaspiro[4.5]decane-2,4-dione, 3-(3-(4-(2-Fluorophenyl) piperazin-1-yl) propyl)-8-phenyl-1,3-diazaspiro[4.5]decane-2,4-dione, 3-(3-(4-(3-Fluorophenyl) piperazin-1-yl) propyl)-8-phenyl-1,3-diazaspiro[4.5]decane-2,4-dione, 3-(3-(4-(4-Fluorophenyl) piperazin-1-yl) propyl)-8-phenyl-1,3-diazaspiro[4.5]decane-2,4-dione, and 3-(3-(4-(4-Fluorobenzoyl) piperazin-1-yl) propyl)-8-phenyl-1,3-diazaspiro[4.5]decane-2,4-dione, or a pharmaceutically acceptable salt, solvate, metabolite, deuterated analogue, derivative, prodrug, or combinations thereof. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior to a compound disclosed herein, such as about three or about one hours prior to administration of a compound disclosed herein. In some embodiments, the serotonin receptor modulator is administered at most about one hour prior to the presently disclosed compound. Thus, in some embodiments of combination therapy with the presently disclosed compounds, the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, the compound of the present application is provided at a dose of from about 10 mg to about 100 mg, or from about 20 mg to about 200 mg, or from about 15 mg to about 300 mg, and the serotonin receptor modulator is provided at a dose of about 10 mg to about 100 mg.

In some embodiments, dopamine receptor modulators, such as modulators of dopamine receptor type 2 (D2 modulators, e.g., D2 agonists), are used to treat a brain disorder. The presently disclosed pharmaceutical compositions of idazoxan may be used in combination with a second therapeutic agent that also is a D2 modulator. In such cases the second therapeutic agent can be an agonist or an antagonist. Dopamine receptor modulators useful as second therapeutic agents for combination therapy as described herein are known to those of skill in the art and include, without limitation, roluperiodone (CYR-101, MIN-101), quetiapine, olanzapine, altanserin, acepromazine, nefazodone, risperidone, trazodone, brexpiprazole, cariprazine, agomelatine, setoperone, zotepine, clozapine, chlorpromazine, sertindole, iloperidone, paliperidone, asenapine, amisulpride, aripiprazole, lurasidone, ziprasidone, lumateperone, perospirone, mosapramine, xanomeline, buspirone, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, an extended-release form of aripiprazole including Abilify Maintena, or a pharmaceutically acceptable salt, solvate, metabolite, deuterated analogue, derivative, prodrug, or combinations thereof. In some embodiments, the dopamine receptor modulator is administered prior to a compound disclosed herein, such as about three or about one hours prior to administration of a compound disclosed herein. In some embodiments, the dopamine receptor modulator is administered at most about one hour prior to the presently disclosed compound. Thus, in some embodiments of combination therapy with the presently disclosed compounds, the second therapeutic agent is a dopamine receptor modulator. In some embodiments the second therapeutic agent dopamine receptor modulator is provided at a dose of from about 10 mg to about 350 mg. In some embodiments, the dopamine receptor modulator is provided at a dose of from about 20 mg to about 200 mg. In some embodiments, the dopamine receptor modulator is provided at a dose of from about 10 mg to about 100 mg. In certain such embodiments, the compound of the present application is provided at a dose of from about 10 mg to about 100 mg, or from about 20 mg to about 200 mg, or from about 15 mg to about 300 mg, and the dopamine receptor modulator is provided at a dose of about 10 mg to about 100 mg.

U.S. Pre-Grant Publication No. 20060281735 and U.S. Pat. No. 5,663,167 disclose the combination of idazoxan and D2 antagonists and are hereby incorporated by reference.

In some embodiments, the pharmaceutical compositions of idazoxan may be used in combination with a second agent to treat Alzheimer's disease. In some embodiments, the pharmaceutical compositions of idazoxan are used in combination with a cholinesterase inhibitor and/or a NMDA receptor antagonist. Exemplary and nonlimiting agents for treating Alzheimer's disease that may be administered in combination with the pharmaceutical compositions of idazoxan include Leqembi (lecanemab-irmb), Donepezil (Aricept), Galantamine (Razadyne), Rivastigmine (Exelon), Memantine (Namenda), Memantine (Namzaric), Ginkgo biloba extract, Aducanumab, 3APS, 3TC, AAB-001, AADvac1, AAV2-BDNF Gene Therapy, ABBV-8E12, ABBV-552, ABBV-916, ABT-089, ABT-126, ABT-288, ABT-384, ABvac40, AC-1204, AC-3933, AC-OLE-01-VA, ACC-001, Acetyl-L-Carnitine, ACI-24.060, ACI-35.030, Acitretin, Active tPBM, ACU193, Acumor XL, AD-35, adenosine triphosphate, Adlarity, aducanumab, Aduhelm, AGB101, Akpha E, AL001, AL002, Albumin 5%, Albumin 20%, Alfoatirin, Allogeneic MSC, Allopregnanolone, ALN-APP, ALZ-101, Alzhok, ALZT-OPla, ALZT-OP1b, amlodipine, AMX0035, AN-1792, ANAVEX2-73, APH-1105, APNmAb005, Aqua Gem-E, Aquasol E, AQW051, AR1001, ARBs, Aricept, Aricept ODT, aripiprazole, armodafinil, astaxanthin, AstroStem, Atabecestat, ATH-1017, atomoxetine hydrochloride, Atorvastatin, Atorvastatin calcium, Atropine, AV-1959D, Avagacestat, AVE1625, AVP-786, AVP-923-30, AXS-05 (dextromethorphan-bupropion), AZD0530, AZD1446, AZD3480, AZD3480, AZD5213, Azeliragon, BAC, Bapineuzumab, Baricitinib, Belsomra, Benfotiamine, Bepranemab, Bexarotene, BI 409306, BI 425809, bifeprunox, BIIB080, BIIB092, BMS-708163, BPDO-1603, BPN14770, Brexpiprazole, Bromocriptine, Bryostatin, Bryostatin 1, Buntanetap, Buprenorphine, C-11 ER-176, CAD106 Immunotherapy, Canakinumab, caprylic triglyceride, caprylidene, Carvedilol, CB-AC-02, CCB, Celecoxib (Celebrex), CERE-110, Cerebrolysin, CHF 5074, Choline alfoscerate, Cholinergic antagonist, Cholinesterase Inhibitor, Cilostazol, Circadin, citalopram, CMS121, CNP520, COR388, CORT108297, CPC-201, Crenezumab, CT1812, CX516, CY6463, DAOI-A, DAOI-B, DAOI-C, Dapagliflozin, Daratumumab Injection, Dasatinib, DCB-AD1, Deferiprone, delta-9-tetrahydrocannabinol, dex-methylphenidate, Dextroamphetamine, DHP1401, Dimebon, Divalproex, Divalproex ER, dl-alpha-tocopherol, Docosahexaenoic Acid, Donanemab, donepezil, donepezil/memantine, Donepezil HCL, Dronabinol, DSPE-DOTA-Gd Liposomal Injection, E-400 Clear, E-600, E-Gems, E2020, E2609, E2814, Ebicomb, Ebixa, EGb761, EHT 0202 etazolate, Elenbecestat (E2609), ELND005, Elontril, Empagliflozin, Emtriva Capsule, ENA713, E Pherol, Epigallocatechin-Gallate, ergoloid mesylates, escitalopram, Escitalopram Oxalate, estrogen, etanercept, ethosuximide, EVP-0962, EVP-6124, EX039, Exelon, Exendin-4 SC, ExPlas, Filgrastim, FK962, Florbetaben (BAY94-9172), Flos gossypii flavonoids, Flutemetamol, Formoterol A, Gabapentin Enacarbil, galantamine, galantamine hydrobromide, Galsya XL, Gantenerumab, Gastro-retentive zinc cysteine, Gatalin XL, gonadorelin acetate, GRF6019, GSK239512, Guanfacine, GV-971, GV1001, Haloperidol, HB-adMSCs, HF0220, Human Mesenchymal Stem Cells and Lactated Riunger's Solution, Huperzine A, Hydergine, Hydralazine hydrochloride, hydrocodone/APAP, Hydroxypropyl Beta Cyclodextrin, IBC-Ab002, icosapent ethyl, ID1201, Idalopirdine, IGC-AD1, Iidalopirdine, Immune Globulin Intravenous (Human), Immunoglobulin, indomethacin, Insulin, Insulin glulisine, IONIS MAPTRx, Isotretinoin, ITI-007, JACI-35.054, JNJ-54861911, JNJ-63733657, KarXT. Prazosin, L-Arginine, L-Serine, ladostigil hemitartrate, Lanabecestat, lecanemab, lecozotan SR, Lemborexant, Lenalidomide, Leqembi, Leuprolide acetate, levetiracetam, Levodopa, Liraglutide, Lithium, Lithium Carbonate, LM11A-31-BHS, LMTM, long-chain triglyceride, Lornoxicam, losartan, Lovostatin, Lu AE58054, Lu AF87908, LX1001, LY450139, LY450139 dihydrate, LY451395, LY2062430, LY2886721, LY3154207, LY3202626, LY3372689, LY3372993, MABT5102A, Masitinib, Masupirdine, MEDI1814 (antiamyloid beta mAb), Medroxyprogesterone, MEM 1003, MEM 3454, memantine, Memantine Hydrochloride, Metformin, Methylene Blue, Methylphenidate, MIB-626, Mifepristone, Minocycline, Mirtazapine, MK-1942, MK-2214, MK-7622, MK0249, MK0677, MK0952, MLC601, MLC901, Modafinil, Montelukast, Montelukast buccal film, MPC-7869, MSDC-0160, MT-4666, MW150, Nabilone, Namenda XR, Namenda, Namzaric, NanoLithium® NP03, Naproxen, Naproxen Sodium (Aleve), NE3107, Nefiracetam, neflamapimod, Nelotanserin, Nemdatine, Neramexane, NewGam 10% IVIG, NGP 555, NIC5-15, Nicotinamide riboside, Nilotinib, Nilotinib BE, Nilvadipine, NIO752, NP031112, NPT 2042, NS 2330, Nuedexta, Obicetrapib, Octagam 10%, Octaplasma, Octohydroaminoacridine Succinate, Olanzapine, Omega 3 PUFA, ONO-2506PO, ORM-12741, ORY-2001, Paracetamol, PBT2, Pepinemab, Perindopril, Perphenazine, PF-01913539, PF-04360365, PF-04447943, PF-04494700, PF-05212377, PF-06648671, Pimavanserin, Pimavanserin tartrate, pioglitazone, piracetam, Piromelatine, PM012, Posiphen, PPI-1019, PQ912, Prednisone, Probucol, Proleukin, PRX-03140, PTI-125, PXT00864, PYM50028, QS-21, Quercetin, Quetiapine, Quetiapine Fumarate, Raloxifene, Ramelteon, Ramipril, Rapamycin, Rasagiline, Razadyne, Razadyne ER, RB-ADSC, Regulatory T cells, REM0046127, Reminyl, Remternetug, reserpine, Resveratrol, Rifaximin, rilapladib, Riluzole, risperidone, rivastigmine, Rivastigmine patch, RO4602522, RO5313534, RO7126209, Rofecoxib, rosiglitazone, Rosiglitazone XR, Rotigotine transdermal patch, RPh201, RVT-101, S-equol, S47445, SAGE-718, SAM-531, SAR110894, Sargramostim, SB-742457, SCI-110, Seltorexant, semagacestat, Semagludtide, Semorinemab, Senicapoc, Sertraline, SGS742, SHR-1707, simufilam, Simvastatin, SK—PC—B70M, SLS-005, SNK01, Sodium oligo-mannurarate, Sodium Oligomannate, Sodium Oxybate, Solanezumab, Solifenacin, Spironolactone, SR57667B, SRA-333, SSR180711C, ST101, sulbutiamine, SUVN-502, suvorexant, T-817MA, T-817MA-H, T-817MA-L, T2: C100, T3D-959, tacrine, Tacrolimus, Talsaclidine, Tamibarotene, Tandospirone Citrate, Tau Mab, TB006, Telmisartan, Tetrahydrobiopterin, TH9507 human growth hormone releasing hormone, Thalidomide, Thiethylperazine, tideglusib, Tilavonemab, TPI 287 (next generation taxane), Tramiprosate, Traneurocin, Transdermal estradiol, Transdermal Methylphenidate, Trazodone, Tricaprilin, troriluzole, TRx0014, TRx0237, Tryptophan, TW001, UB-311, UCMSCs, Valaciclovir/Valacyclovir, Valproate, Varenicline, Venlafaxine, Verubecestat, VI-1121, vitamin e, VP4896, VT301, VX-745, xaliproden (SR57746A), Xanamem, XPro1595, Zagotenemab, Zolpidem, Zoplicone, Zydena

In some embodiments, monoclonal or polyclonal antibodies, such as antibodies against beta amyloid, are used to treat a brain disorder. The presently disclosed pharmaceutical compositions of idazoxan may be used in combination with a second therapeutic agent that also is a monoclonal or polyclonal antibody. For example, the presently disclosed pharmaceutical compositions of idazoxan may be used in combination with Aducanumab or Leqembi (lecanemab-irmb).

### Examples

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort can be had to various other embodiments, modifications, and equivalents thereof which can suggest themselves to those skilled in the art without departing from the spirit of the present disclosure and/or scope of the appended claims.

### NMR Analysis

Analyses (¹H, ¹³C and ¹⁹F NMR) were conducted on a 400 MHz NMR spectrometer using deuterated chloroform, deuterated methanol or deuterated dimethyl sulfoxide as solvent. The chemical shift (d) of each signal was measured in parts per million (ppm) relative the residual solvent peak, and the multiplicity reported together with the associated coupling constant (J), where applicable. Data are reported as (br=broad, s=singlet, d=doublet, t=triplet, q=quartet, m=multiplet).

### Agilent LC-MS Analysis Methodology

Instrument: Agilent 1260 infinity HPLC with Agilent 6130 single quadrupole mass spec.

### Column: Phenomenex Kinetex XB-C₁₈, 50×4.6 mm, 2.6 μm

### Elution Profile

| | % Aqueous (A) | % Organic (B) |
|---|---|---|
| Time (Minutes) | (0.1% Formic Acid in Water) | (100% Acetonitrile) |
| 0 | 95 | 5 |
| 1.37 | 2 | 98 |
| 1.60 | 2 | 98 |
| 1.83 | 95 | 5 |
| 2.25 | 95 | 5 |

Flow rate: 2 mL/min; Detector wavelength: 225+50 nm bandwidth; Column temperature: 40° C.; Injection volume: 1 μl; Mass spec parameters: Scanning in ES+/− & APCI over 70-1000m/z; Needle wash: MeOH wash in vial 4, autosampler set up to do 5 needle washes (to wash the outside of the needle prior to injecting the sample); Sample preparation: 0.5-1.0 mg/ml in either acetonitrile or DMSO depending on the nature of the sample in terms of solubility.

### Waters Alliance LC-MS Analysis Methodology

Acidic method (named as Formic_8 min)
Instrument: Waters 2795 Alliance HPLC system equipped with a 2996 PDA detector
and Micromass ZQ mass spectrometer detector.
Column: Gemini C18, 5 μm, 110 Å, 50×4.6 mm ID
Mobile phase A: 0.1% Formic acid in water
Mobile phase B: 100% acetonitrile

Gradient program (overall run time per injection is 8 minutes):

| Time | Mobile phase A | Mobile phase B |
|---|---|---|
| 0.0 | 95 | 5 |
| 0.6 | 95 | 5 |
| 6.50 | 2 | 98 |
| 7.50 | 2 | 98 |
| 7.60 | 95 | 5 |
| 8.00 | 95 | 5 |

Flow rate: 1 ml/min; Injection volume: 10 μl; Column oven temperature: 40° C.; Detector: PDA UV at 190-400 nm, also fixed λ at 225 nm; Mass spec parameters: MS scan in ES+, ES−, ranging from M/Z 100-1000; Purge solvent involved in injection.

### Example 1: 2-(2,3-dihydrobenzo[b][1,4]dioxin-2-yl-5,6,7,8-d₄)-4,5-dihydro-1H-imidazole (Compound 4)

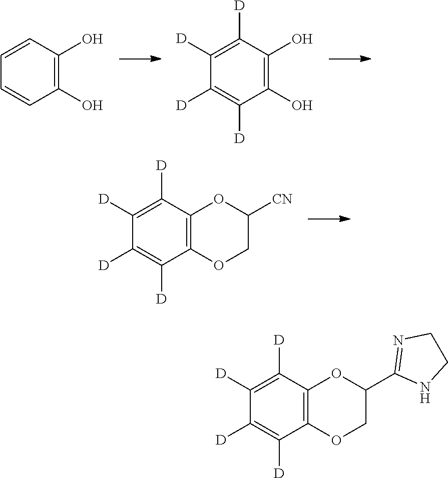 [SMILES: Oc1ccccc1O ; [2H]c1c([2H])c([2H])c(O)c(O)c1[2H] ; [2H]c1c([2H])c([2H])c2c(c1[2H])OCC(C#N)O2 ; [2H]c1c([2H])c([2H])c2c(c1[2H])OCC(C1=NCCN1)O2]

### Step 1: (3,4,5,6-d₄)-benzene-1,2-diol

To a suspension of benzene-1,2-diol (1.00 g, 9.08 mmol) in deuterated water (20.0 mL, 1108 mmol) in a 10-20 mL Biotage microwave vial was added potassium tert-butoxide (510 mg, 4.54 mmol) in one portion with stirring. The vial was placed under an atmosphere of N₂ and sealed, then heated to 140° C. for 15 h (Biotage Initiator microwave). The mixture was extracted with EtOAc (5×50 mL) and the combined organic layers concentrated under reduced pressure to give an oil that solidified on standing. The crude product was triturated with hexane, isolated by filtration, and washed with hexane to afford the title compound (600 mg, 57%) as a solid. Retention time 1.079 mins; m/z=[M−H]⁺ calculated for C₆H₂D₄O₂ 113.0; found 113.0; 1H NMR (400 MHZ, CDCl₃) δ 5.14 (2H, s). The deuterium incorporation of this compound was determined as 97% by quantitative NMR using maleic acid as a reference standard.

### Step 2: 2,3-dihydrobenzo[b][1,4]dioxine-2-carbonitrile-5,6,7,8-d₄

To a solution of (3,4,5,6-d₄)-benzene-1,2-diol (600 mg, 5.26 mmol) and K₂CO₃ (1.45 g, 10.5 mmol) in acetone (12 ml) was added 2-chloroprop-2-enenitrile (460 mg, 5.26 mmol) in one portion. The mixture was heated to 50° C. and stirred for 18 h. The mixture was concentrated under reduced pressure and the brown viscous residue partitioned between H₂O (100 mL) and Et₂O (100 mL). The organic layer was dried over MgSO₄, filtered and the filtrate was concentrated under reduced pressure. The residue was dry loaded on to silica gel then purified by column chromatography on silica gel (10 g Biotage HC cartridge) using DCM (CH₂Cl₂) in hexane gradient (1:9 to 4:6) as eluent to afford the title compound (450 mg, 51%) as a viscous oil. Retention time 1.570 mins; ¹H NMR (400 MHZ, CDCl₃) δ 5.10 (1H, dd, J=4.0 Hz and 2.8 Hz), 4.42 (1H, dd, J=12.0 Hz and 4.0 Hz), 4.35 (1H, dd, J=11.6 Hz and 2.8 Hz).

### Step 3: 2-(2,3-dihydrobenzo[b][1,4]dioxin-2-yl-5,6,7,8-d₄)-4,5-dihydro-1H-imidazole

To a stirred solution of 2,3-dihydrobenzo[b][1,4]dioxine-2-carbonitrile-5,6,7,8-d₄ (144 mg, 0.87 mmol) in dry MeOH (3 mL) with ice bath cooling (0-5° C.) under an atmosphere of N₂ was added NaOMe (1.88 mg, 0.035 mmol) in one portion. The mixture was warmed to rt and stirred for 6 h, then cooled in an ice bath and ethane-1,2-diamine (64 μL, 0.96 mmol) was added in one portion. After 5 mins, HCl in MeOH (1.25M; 767 μL, 0.96 mmol) was added dropwise. The mixture was allowed to warm to rt and was stirred overnight. The solvent was concentrated under reduced pressure and the residue was purified by column chromatography on silica gel using 2M ammonia in MeOH/DCM (CH₂Cl₂) (1:9) as eluent to afford the title compound (72 mg, 39%) as a solid. Retention time 0.994 mins; m/z=[M+H]⁺ calculated for C₁₁H₈D₄N₂O₂ 209.1; found 209.2; 1H NMR (400 MHZ, CDCl₃) δ 5.06 (1H, br s), 4.89 (1H, dd, J=7.6 Hz and 2.4 Hz), 4.54 (1H, dd, J=11.6 Hz and 2.8 Hz), 4.18 (1H, dd, J=7.6 Hz and 11.2 Hz), 4.10-3.25 (4H, br m). The deuterium incorporation of this compound was determined as 96.3% by measurement of the integrals of residual aromatic protons in the ¹H NMR.

### Example 2: 2-(2,3-dihydrobenzo[b][1,4]dioxin-2-yl-5,6,7,8-d4)-4,5-dihydro-1H-imidazole-4,4,5,5-d4 (Compound 52)

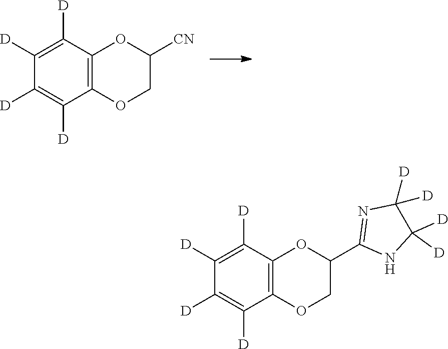 [SMILES: [2H]c1c([2H])c([2H])c2c(c1[2H])OCC(C#N)O2 ; [2H]c1c([2H])c([2H])c2c(c1[2H])OCC(C1=NC([2H])([2H])C([2H])([2H])N1)O2]

To a stirred solution of 5,6,7,8-tetradeuterio-2,3-dihydro-1,4-benzodioxine-3-carbonitrile (139 mg, 0.84 mmol) in dry MeOH (3 mL) with ice bath cooling (0-5° C.) under an atmosphere of N₂ was added sodium methoxide (25.0 wt % solution in MeOH, 7.70 μL, 0.034 mmol) in one portion. The mixture was warmed to rt and stirred for 6 h, then cooled in an ice bath and 1,1,2,2-tetradeuterioethane-1,2-diamine (61.8 μL, 0.93 mmol) was added in one portion. After 5 mins, HCl in MeOH (1.25M in MeOH, 741 μL, 0.93 mmol) was added dropwise. The mixture was allowed to warm to rt and was stirred overnight, then H₂O added (40 mL), basified with 33-34% aqueous NH₃ solution (5 mL) and extracted with CHCl₃ (2×20 mL). The combined organic layers were concentrated under reduced pressure to afford the title compound (155 mg, 77%) as a solid. Retention time 0.957 mins; m/z=[M+H]⁺ calculated for C₁₁H₄D₈N₂O₂, 213.1; found 213.2; ¹H NMR (400 MHZ, CDCl₃) δ 5.03 (1H, br s), 4.89 (1H, dd, J=8.0 Hz and 2.8 Hz), 4.54 (1H, dd, J=11.6 Hz and 2.8 Hz), 4.18 (1H, dd, J=11.2 Hz and 7.6 Hz).

### Example 3: 2-(2,3-dihydrobenzo[b][1,4]dioxin-2-yl-6-d)-4,5-dihydro-1H-imidazole (Compound 1)

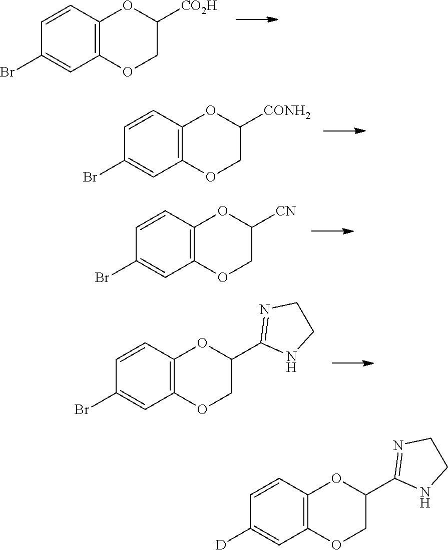

### Step 1: 6-bromo-2,3-dihydro-1,4-benzodioxine-2-carboxamide

To a stirred suspension of 6-bromo-2,3-dihydro-1,4-benzodioxine-2-carboxylic acid (250 mg, 0.97 mmol) in toluene (5 mL) was added thionyl chloride (141 μL, 1.93 mmol) in one portion. The suspension was heated to 100° C. and stirred for 2 h, then the toluene was removed under reduced pressure. The residue was co-evaporated with toluene (2×10 mL) to remove excess thionyl chloride. The residue was suspended in 1,4-dioxane (5 mL) and this mixture was added dropwise over 5 min to stirred 33-34% aqueous NH₃ solution (5 mL) with ice bath cooling. The ice bath was removed, and the mixture was warmed to rt and stirred for 30 min, then H₂O (40 mL) was added. The emerging precipitate was collected by filtration and air dried at 40° C. to afford the title compound (224 mg, 89%) as a solid. Retention time 1.510 mins; ¹H NMR (400 MHZ, DMSO-d₆) δ 7.53 (1H, br. s), 7.48 (1H, br. s), 7.07 (1H, d, J=2.4 Hz), 7.03 (1H, dd, J=8.4 Hz, 2.4 Hz), 6.92 (1H, d, J=8.8 Hz), 4.79-4.47 (1H, m), 4.36-4.24 (2H, m).

### Step 2:6-Bromo-2,3-Dihydro-1,4-Benzodioxine-2-Carbonitrile

To a stirred suspension of 6-bromo-2,3-dihydro-1,4-benzodioxine-2-carboxamide (170 mg, 0.66 mmol) in DMF (4 mL) at rt was added 2,4,6-trichloro-1,3,5-triazine (46 mg, 0.26 mmol) portion-wise over 2 min. The mixture was stirred at rt for 18 h, then a further portion of 2,4,6-trichloro-1,3,5-triazine (46 mg, 0.26 mmol) was added in one portion and the mixture was stirred at rt overnight. The mixture was combined with that of a smaller validation batch (50 mg scale), then poured into H₂O (50 mL) and extracted with Et₂O (4×20 mL). The combined organic layers were concentrated under reduced pressure and the residue was purified by column chromatography on silica gel (Biotage Isolera system; 5 g HC silica cartridge) using EtOAc in hexane gradient (1:9 to 4:6) as eluent to afford the title compound (176 mg, 85%) as a solid. Retention time 1.171 mins; 1H NMR (400 MHZ, CDCl₃) δ 7.12 (1H, J=2.0 Hz), 7.04 (1H, dd, J=8.8 Hz and 2.4 Hz), 6.84 (1H, J=8.4 Hz), 5.13-5.09 (1H, m), 4.49-4.30 (2H, m).

### Step 3: 2-(6-bromo-2,3-dihydro-1,4-benzodioxin-2-yl)-4,5-dihydro-1H-imidazole

To a stirred solution of 6-bromo-2,3-dihydro-1,4-benzodioxine-2-carbonitrile (175 mg, 0.73 mmol) in dry MeOH (4 mL) with ice bath cooling (0-5° C.) under an atmosphere of N₂ was added NaOMe (25 wt % solution in MeOH, 6.67 μL, 0.029 mmol) in one portion. The mixture was warmed to rt and stirred for 6 h, at which point TLC analysis (eluent 1:1 hexane-CH₂Cl₂) indicated that virtually all the starting nitrile had been converted to the imidate. The mixture was cooled in an ice bath and ethane-1,2-diamine (53.5 μL, 0.80 mmol) was added in one portion. After 5 min, HCl in MeOH (1.25 M; 642 μL, 0.80 mmol) was added dropwise. The mixture was allowed to warm to rt and was stirred overnight, then H₂O (40 mL) was added, the mixture was basified with 33-34% aqueous NH₃ solution (5 mL) and extracted with DCM (CH₂Cl₂) (3×20 mL). The combined organic layers were dried over Na₂SO₄, filtered and the filtrate was concentrated under reduced pressure to afford the title compound (169 mg, 81%) as a solid. Retention time 1.172 mins; m/z=[M+H]⁺ calculated for C₁₁H₁₁BrN₂O₂ 283.0; found 283.0; 1H NMR (400 MHZ, CDCl₃) δ 7.06 (1H, d, J=2.0 Hz), 6.97 (1H, dd, J=8.8 Hz and 2.4 Hz), 6.78 (1H, J=8.8 Hz), 5.29 (1H, br s), 4.85 (1H, dd, J=7.6 Hz and 2.4 Hz), 4.53 (1H, dd, J=11.6 Hz and 2.4 Hz), 4.22-4.13 (1H, m), 3.97-3.81 (2H, br m), 3.57-3.38 (2H, br m).

### Step 4: 2-(2,3-dihydrobenzo[b][1,4]dioxin-2-yl-6-d)-4,5-dihydro-1H-imidazole

A mixture of 2-(6-bromo-2,3-dihydro-1,4-benzodioxin-2-yl)-4,5-dihydro-1H-imidazole (135 mg, 0.48 mmol) and 10% Pd/C (45 mg, 0.042 mmol) in MeOH-d₄ (3.5 mL) was stirred under an atmosphere of deuterium (ca. 20 psi) for 2 h at rt. The reaction mixture was filtered to remove Pd/C catalyst and combined with that of a validation batch (30 mg scale). The reaction mixture was added to water (40 mL), basified with 33-34% aqueous NH₃ solution (5 mL) and extracted with CHCl₃ (2×20 mL). The combined organic layers were concentrated under reduced pressure and the crude residue was purified by column chromatography on silica gel using 2M NH₃ in MeOH/DCM (CH₂Cl₂) (1:9) as eluent to afford the title compound (60 mg, 50%) as a viscous oil. Retention time 0.959 mins; m/z=[M+H]⁺ calculated for C₁₁H₁₁DN₂O₂ 206.1; found 206.2; 1H NMR (400 MHZ, CDCl₃) δ 6.99-6.86 (3H, m), 4.94 (0.65H due to partial deuteration of this position, dd, J=7.2 Hz and 2.4 Hz), 4.58-4.51 (1H, m), 4.27-4.19 (1H, m), 3.69 (4H, s).

### Example 4: 2-(2,3-dihydrobenzo[b][1,4]dioxin-2-yl-7-d)-4,5-dihydro-1H-imidazole (Compound 2)

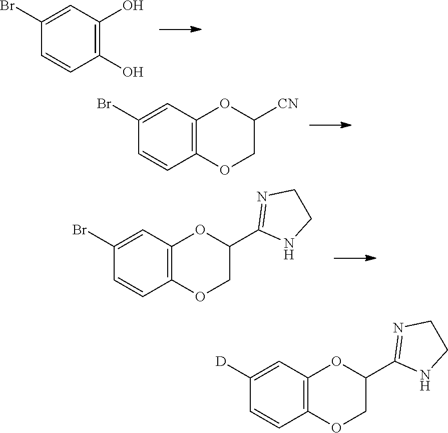

### Step 1: 7-Bromo-2,3-dihydro-1,4-benzodioxine-2-carbonitrile

To a solution of 4-bromobenzene-1,2-diol (1.0 g, 5.29 mmol) and K₂CO₃ (1.46 g, 10.6 mmol) in acetone (20 mL) was added 2-chloroprop-2-enenitrile (463 mg, 5.29 mmol) in one portion. The mixture was heated to 85° C. under microwave irradiation (Biotage Initiator microwave) and stirred for 12 h. The mixture was concentrated under reduced pressure and the residue was partitioned between H₂O (100 mL) and EtOAc (100 mL). The organic layer was concentrated under reduced pressure and the residue combined with the crude product of a validation batch (1 g scale), then purified by column chromatography on silica gel (Biotage Isolera system; 10 g HC silica cartridge) using DCM (CH₂Cl₂)/hexane (1:9 to 4:6 gradient) as eluent to afford the title compound (703 mg, 27%) as a solid. Retention time 1.711 min; 1H NMR (400 MHZ, CDCl₃) δ 7.12 (1H, d, J=2.4 Hz), 7.10-7.01 (1H, m), 6.84 (1H, d, J=8.8 Hz), 5.12-5.06 (1H, m), 4.42 (1H, dd, J=11.6 Hz and 3.6 Hz), 4.39-4.30 (1H, m).

### Step 2: 2-(7-Bromo-2,3-dihydro-1,4-benzodioxin-2-yl)-4,5-dihydro-1H-imidazole

To a stirred solution of 7-bromo-2,3-dihydro-1,4-benzodioxine-2-carbonitrile (505 mg, 2.10 mmol) in dry MeOH (10 mL) with ice bath cooling (0-5° C.) under an atmosphere of N₂ was added NaOMe (4.55 mg, 0.084 mmol) in one portion. The mixture was warmed to rt and stirred for 6 h, then cooled in an ice bath and ethane-1,2-diamine (0.155 mL, 2.31 mmol) was added in one portion. After 5 min, HCl in MeOH (1.25M; 1.85 mL, 2.31 mmol) was added dropwise. The mixture was allowed to warm to rt and was stirred overnight, then H₂O (100 mL) added, basified with 33-34% aqueous NH₃ solution (10 mL) and extracted with DCM (CH₂Cl₂) (3×40 mL). The organic layers were dried over Na2SO4, filtered, the filtrate was concentrated under reduced pressure and combined with that of a validation batch (200 mg scale). The crude residue was purified by column chromatography on silica gel using 2M ammonia in MeOH/DCM (CH₂Cl₂) (1:9) as eluent to afford the title compound (331 mg, 39%) as a solid. Retention time 1.169 min; m/z=[M+H]⁺ calculated for C₁₁H₁₁BrN₂O₂ 283.0; found 283.0; ¹H NMR (400 MHZ, CD₃OD) δ 7.12 (1H, d, J=2.4 Hz), 6.97 (1H, dd, J=8.8 Hz and 2.4 Hz), 6.80 (1H, d, J=8.4 Hz), 4.92-4.86 (1H, m), 4.45-4.38 (1H, m), 4.20-4.12 (1H, m), 3.63 (4H, s).

### Step 3: 2-(2,3-dihydrobenzo[b][1,4]dioxin-2-yl-7-d)-4,5-dihydro-1H-imidazole

To a mixture of 2-(7-bromo-2,3-dihydro-1,4-benzodioxin-2-yl)-4,5-dihydro-1H-imidazole (30 mg, 0.11 mmol) and 10% Pd/C (10 mg) in MeOH-d4 (0.75 mL) at rt was stirred under an atmosphere of deuterium (ca. 20 psi) for 2 h. The mixture was filtered to remove Pd/C and the filtrate was loaded directly onto silica wetted with 2M NH₃ in MeOH/DCM (CH₂Cl₂) (1:9) and purified by column chromatography on silica gel using 2M NH₃ in MeOH/DCM (CH₂Cl₂) (1:9) as eluent to afford the desired product (18 mg, 82%) as a viscous oil. Retention time 0.986 min; m/z=[M+H]⁺ calculated for C₁₁H₁₁DN₂O₂ 206.0; found 206.2; ¹H NMR (400 MHz, CD₃OD) δ 6.94 (1H, br s), 6.85 (2H, br s), 4.91-4.87 (1H, m, partially obscured by water peak), 4.46-4.38 (1H, m), 4.20-4.10 (1H, m), 3.64 (4H, s).

### Example 5: 2-(2,3-dihydrobenzo[b][1,4]dioxin-2-yl-6,7-d2)-4,5-dihydro-1H-imidazole (Compound 3)

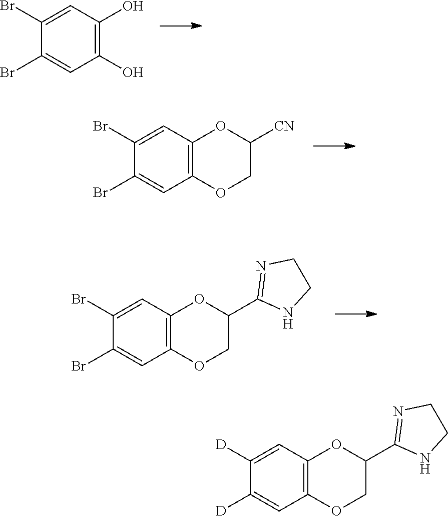 [SMILES: Oc1cc(Br)c(Br)cc1O ; N#CC1COc2cc(Br)c(Br)cc2O1 ; Brc1cc2c(cc1Br)OC(C1=NCCN1)CO2 ; [2H]c1cc2c(cc1[2H])OC(C1=NCCN1)CO2]

### Step 1: 6,7-Dibromo-2,3-dihydro-1,4-benzodioxine-2-carbonitrile

To a stirred solution of 4,5-dibromobenzene-1,2-diol (1.53 g, 5.71 mmol) in acetone (30 mL) was added 2-chloroprop-2-enenitrile (0.50 g, 5.71 mmol) in one portion. The mixture was heated to 50° C. and stirred overnight, then concentrated under reduced pressure and the residue partitioned between H₂O (150 mL) and EtOAc (150 mL). The organic layer was concentrated under reduced pressure and the residue was purified by column chromatography on silica gel (10 g Biotage HC cartridge) using DCM (CH₂Cl₂) in hexane (2:8 to 4:6) as eluent to afford the title compound (1.06 g, 58%) as a solid. Retention time 1.765 min; m/z=[M+H]⁺ calculated for C₉H₅Br₂NO₂ 317.8; found 317.8; ¹H NMR (400 MHZ, CDCl₃) δ 7.15-7.08 (2H, m), 5.24 (1H, dd, J=3.6 Hz and 2.4 Hz), 4.56 (1H, dd, J=12.0 Hz and 3.6 Hz), 4.43 (1H, dd, J=12.0 Hz and 2.4 Hz)

### Step 2: 2-(6, 7-Dibromo-2,3-dihydro-1,4-benzodioxin-2-yl)-4,5-dihydro-1H-imidazole

To a stirred solution of 6,7-dibromo-2,3-dihydro-1,4-benzodioxine-2-carbonitrile (420 mg, 1.32 mmol) in dry MeOH (16 mL) under an atmosphere of N₂ with ice bath cooling (0-5° C.) was added sodium methoxide (25 wt % solution in MeOH, 12 μL, 0.053 mmol) in one portion. The mixture was warmed to rt and stirred for 6 h, then cooled in an ice bath and ethane-1,2-diamine (96.7 μL, 1.45 mmol) was added in one portion. After 5 mins, 1.25M HCl in MeOH (1.16 mL, 1.45 mmol) was added dropwise. The mixture was allowed to warm to rt and was stirred overnight. The precipitate was isolated by filtration, washed with cold MeOH (1 mL) and air dried to afford the title compound (312 mg, 62%) as a solid. Retention time 1.213 mins; m/z=[M+H]⁺ calculated for C₁₁H₁₀Br₂N₂O₂ 362.9; found 362.8; 1H NMR (400 MHZ, DMSO-d₆) δ 7.14-7.08 (2H, m), 6.50 (1H, br. s), 5.03 (1H, dd, J=6.4, 2.4 Hz), 4.52 (1H, dd, J=11.6, 2.8 Hz), 4.34 (1H, dd, J=11.6, 6.4 Hz), 3.80-3.20 (4H, br. s).

### Step 3: 2-(2,3-dihydrobenzo[b][1,4]dioxin-2-yl-6,7-d2)-4,5-dihydro-1H-imidazole

To a stirred solution of 2-(6,7-dibromo-2,3-dihydro-1,4-benzodioxin-2-yl)-4,5-dihydro-1H-imidazole (303 mg, 0.84 mmol) and 10% Pd/C (79 mg, 0.74 mmol) in MeOH-d₄ (6 mL) was stirred under an atmosphere of D₂ (ca. 20 psi) at rt for 24 h. The mixture was filtered and the filtrate was concentrated under reduced pressure. The residue was purified by column chromatography on silica gel using 2M NH₃ in MeOH/DCM (CH₂Cl₂) (5:95 to 15:85) as eluent to afford the title compound (126 mg, 73%) as a viscous oil. Retention time 0.753 mins; m/z=[M+H]⁺ calculated for C₁₁H₁₀D₂N₂O₂ 207.1; found 207.2; ¹H NMR (400 MHZ, CD₃OD) δ 6.93-6.85 (2H, m), 5.42 (1H, dd, J=5.6, 2.4 Hz), 4.47 (1H, dd, J=12.0, 2.8 Hz), 4.38 (1H, dd, J=12.0, 5.6 Hz), 4.00 (4H, s).

### Example 6: 2-(2,3-dihydrobenzo[b][1,4]dioxin-2-yl-6-d)-4,5-dihydro-1H-imidazole-4,4,5,5-d4 (Compound 49)

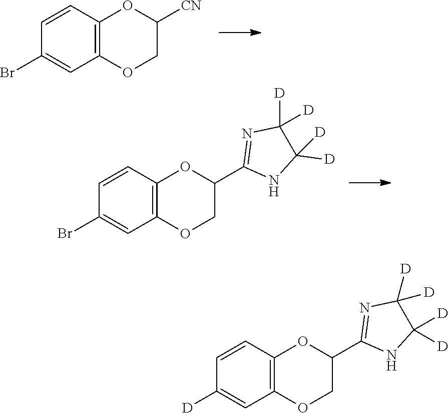 [SMILES: N#CC1COc2cc(Br)ccc2O1 ; [2H]C1([2H])N=C(C2COc3cc(Br)ccc3O2)NC1([2H])[2H] ; [2H]c1ccc2c(c1)OCC(C1=NC([2H])([2H])C([2H])([2H])N1)O2]

### Step 1: 2-(6-bromo-2,3-dihydrobenzo[b][1,4]dioxin-2-yl)-4,5-dihydro-1H-imidazole-4,4,5,5-d4

To a stirred mixture of 6-bromo-2,3-dihydro-1,4-benzodioxine-2-carbonitrile (170 mg, 0.71 mmol) in dry MeOH (4 ml) under an atmosphere of N₂ with ice bath cooling (0-5° C.) was added sodium methanolate (25 wt % solution in MeOH, 6.5 μL, 0.028 mmol) in one portion. The mixture was warmed to rt and stirred for 7 h, then cooled in an ice bath and 1,1,2,2-tetradeuterioethane-1,2-diamine (48.9 μL, 0.78 mmol) was added in one portion. After 5 mins, hydrogen chloride (1.25M in MeOH) (623 μL, 0.78 mmol) was added dropwise. The mixture was allowed to warm to rt and was stirred overnight, then poured into H₂O (40 mL), basified with 33-34% aqueous NH₃ solution (5 mL) and the emerging precipitate was isolated by filtration to afford the title compound (134 mg, 64%) as a solid after air drying. Retention time 2.850 mins; m/z=[M+H]⁺ calculated for C₁₁H₇D₄BrN₂O₂ 287.0; found 287.0; ¹H NMR (400 MHZ, CDCl₃) δ 7.06 (1H, d, J=2.4 Hz), 6.97 (1H, dd, J=8.4, 2.4 Hz), 6.78 (1H, J=8.4 Hz), 4.98 (1H, br. s), 4.85 (1H, dd, J=7.6, 2.4 Hz), 4.53 (1H, dd, J=11.6, 2.8 Hz), 4.17 (1H, dd, J=11.6, 8.0 Hz).

### Step 2: 2-(2,3-dihydrobenzo[b][1,4]dioxin-2-yl-6-d)-4,5-dihydro-1H-imidazole-4,4,5,5-d4

To a stirred mixture 2-(6-bromo-2,3-dihydro-1,4-benzodioxin-2-yl)-4,5-dihydro(4,4,5,5-²H₄)-1H-imidazole (130 mg, 0.45 mmol) and 10% Pd/C (42.7 mg, 0.04 mmol) in MeOH-d4 (2.5 mL) was stirred under an atmosphere of D2 (ca. 20 psi) at rt for 2 h. The mixture was filtered, poured into H₂O (40 mL), basified with 33-34% aqueous NH₃ solution (5 mL) and extracted with CHCl₃ (2×20 mL). The combined organic layers were concentrated under reduced pressure and the crude residue was purified by column chromatography on silica gel using 2M NH₃ in MeOH/DCM (CH₂Cl₂) (1:9) as eluent to afford the title compound (78 mg, 82%) as a viscous oil. Retention time 0.779 mins; m/z=[M+H]⁺ calculated for C₁₁H₇D₅N₂O₂ 210.1; found 210.2; 1H NMR (400 MHz, CDCl₃) δ 7.00-6.84 (3H, m), 4.90 (0.57H due to partial deuteration of this position, dd, J=8.0, 2.8 Hz), 4.57-4.50 (1H, m), 4.22-4.13 (1H, m).

### Example 7: 2-(2,3-dihydrobenzo[b][1,4]dioxin-2-yl-7-d)-4,5-dihydro-1H-imidazole-4,4,5,5-d4 (Compound 50)

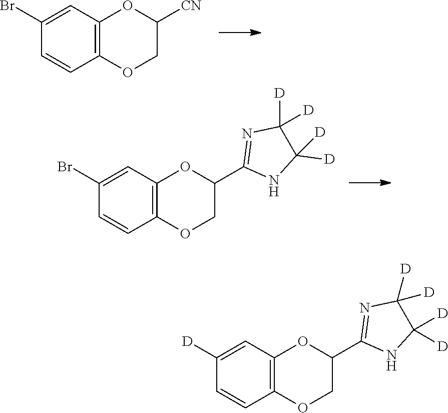 [SMILES: N#CC1COc2ccc(Br)cc2O1 ; [2H]C1([2H])N=C(C2COc3ccc(Br)cc3O2)NC1([2H])[2H] ; [2H]c1ccc2c(c1)OC(C1=NC([2H])([2H])C([2H])([2H])N1)CO2]

### Step 1: 2-(7-bromo-2,3-dihydrobenzo[b][1,4]dioxin-2-yl)-4,5-dihydro-1H-imidazole-4,4,5,5-d4

To a mixture of 7-bromo-2,3-dihydro-1,4-benzodioxine-2-carbonitrile (200 mg, 0.88 mmol) in dry MeOH (4 mL) under an atmosphere of N₂ with ice bath cooling (0-5° C.) was added sodium methoxide solution in MeOH (25 wt %, 7.6 μl, 0.033 mmol) in one portion. The mixture was warmed to rt and stirred for 6 h, then cooled in an ice bath and 1,1,2,2-tetradeuterioethane-1,2-diamine (57.5 μL, 0.92 mmol) was added in one portion. After 5 mins, 1.25M HCl in MeOH (0.73 mL, 0.92 mmol) was added dropwise. The mixture was allowed to warm to rt and was stirred overnight, then diluted with H₂O (40 mL), basified with 33-34% aqueous NH₃ solution (5 mL) and extracted with DCM (CH₂Cl₂) (3×20 mL). The combined organic layers were dried over Na₂SO₄, filtered and the filtrate was concentrated under reduced pressure to afford the title compound (210 mg, 87%) as a solid. Retention time 1.169 mins; m/z=[M+H]⁺ calculated for C₁₁H₇D₄BrN₂O₂ 287.0; found 287.0; ¹H NMR (400 MHZ, DMSO-d₆) δ 7.12-7.08 (1H, m), 7.05-6.98 (1H, m), 6.90-6.81 (1H, m), 4.92-4.86 (1H, m), 4.46-4.37 (1H, m), 4.26-4.15 (1H, m).

### Step 2: 2-(2,3-dihydrobenzo[b][1,4]dioxin-2-yl-7-d)-4,5-dihydro-1H-imidazole-4,4,5,5-d4

To a mixture of 2-(7-bromo-2,3-dihydrobenzo[b][1,4]dioxin-2-yl)-4,5-dihydro-1H-imidazole-4,4,5,5-d4 (205 mg, 0.71 mmol) and 10% Pd/C (67 mg) in MeOH-d₄ (4 mL) was stirred under an atmosphere of D₂ (ca. 20 psi) at rt for 2 h. The mixture was filtered and the filtrate was loaded directly onto silica wetted with 2M NH₃ in MeOH/DCM (CH₂Cl₂) (1:9). The mixture was purified by column chromatography on silica gel using 2M NH₃ in MeOH/DCM (CH₂Cl₂) (1:9) as eluent to afford the title product (86 mg, 57%) as a viscous oil. Retention time 0.981 mins; m/z=[M+H]⁺ calculated for C₁₁H₇D₅N₂O₂ 210.1; found 210.2; ¹H NMR (400 MHZ, CDCl₃) δ 6.96-6.82 (3H, m), 5.02 (1H, br. s), 4.88 (0.62H due to partial deuteration of this position, dd, J=8.0, 5.2 Hz), 4.57-4.50 (1H, m), 4.21-4.11 (1H, m).

### Example 8: 2-(2,3-dihydrobenzo[b][1,4]dioxin-2-yl-6,7-d2)-4,5-dihydro-1H-imidazole-4,4,5,5-d4 (Compound 51)

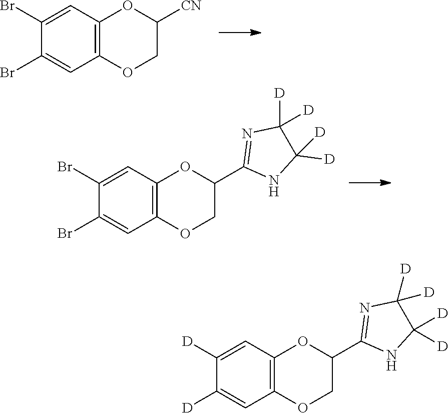 [SMILES: N#CC1COc2cc(Br)c(Br)cc2O1 ; [2H]C1([2H])N=C(C2COc3cc(Br)c(Br)cc3O2)NC1([2H])[2H] ; [2H]c1cc2c(cc1[2H])OC(C1=NC([2H])([2H])C([2H])([2H])N1)CO2]

### Step 1: 2-(6,7-dibromo-2,3-dihydrobenzo[b][1,4]dioxin-2-yl)-4,5-dihydro-1H-imidazole-4,4,5,5-d4

To a stirred solution of 6,7-dibromo-2,3-dihydro-1,4-benzodioxine-3-carbonitrile (86 mg, 0.27 mmol) in dry MeOH (4 mL) under an atmosphere of N₂ with ice/water bath cooling was added sodium methoxide (25% wt in MeOH, 2.5 μL, 0.011 mmol). The mixture was warmed to rt and stirred for 18 h, then cooled in an ice bath and 1, 1,2,2-tetradeuterioethane-1,2-diamine (18.6 μL, 0.30 mmol) was added in one portion. After 5 mins, 1.25M HCl in MeOH (237 μL, 0.30 mmol) was added dropwise. The mixture was allowed to warm to rt and was stirred for 24 h, then added to ice water (30 mL) containing 33-34% aqueous NH₃ solution (5 mL). The mixture was stirred for 5 min, and the emerging precipitate was isolated by filtration and air dried to afford the title compound (32 mg, 32%) as a solid. Retention time 1.192 mins; m/z=[M+H]⁺ calculated for C₁₁H₆Br₂D₄N₂O₂ 366.9; found 366.9; ¹H NMR (400 MHz, DMSO-d₆) δ 7.17-7.08 (2H, m), 5.04 (1H, dd, J=6.8, 2.8 Hz), 4.53 (1H, dd, J=11.6, 2.8 Hz), 4.35 (1H, dd, J=11.6, 6.8 Hz).

### Step 2: 2-(2,3-dihydrobenzo[b][1,4]dioxin-2-yl-6,7-d2)-4,5-dihydro-1H-imidazole-4,4,5,5-d4

To a stirred mixture of 2-(6,7-dibromo-2,3-dihydrobenzo[b][1,4]dioxin-2-yl)-4,5-dihydro-1H-imidazole-4,4,5,5-d4 (30 mg, 0.082 mmol) and 10% Pd/C (15 mg, 0.014 mmol) in MeOH-d₄ (2.5 ml) was added N-ethyl-N-isopropyl-propan-2-amine (28 mL, 0.16 mmol). The mixture was stirred under an atmosphere of D₂ (ca. 20 psi) at rt for 24 h, then filtered through a pad of Celite. The filtrate was diluted with H₂O (40 mL), basified with 33-34% aqueous NH₃ solution (5 mL) and extracted with CHCl₃ (2×20 ml). The combined organic layers were concentrated under reduced pressure to afford the title compound (11 mg, 63%) as a viscous oil. Retention time 0.935 mins; m/z=[M+H]⁺ calculated for C₁₁H₆D₆N₂O₂ 211.1; found 211.2; ¹H NMR (400 MHz, CDCl₃) δ 6.92-6.85 (2H, m), 5.02 (1H, br. s), 4.88 (ca. 0.25H, dd, J=7.6, 2.4 Hz), 4.58-4.49 (1H, m), 4.22-4.14 (1H, m).
NB-LCMS and NMR indicate ca. 75% deuteration of the CH α to the imidazoline ring.

### Example 9: 2-(2,3-dihydrobenzo[b][1,4]dioxin-2-yl)-4,5-dihydro-1H-imidazole-4,4,5,5-d4 (Compound 64)

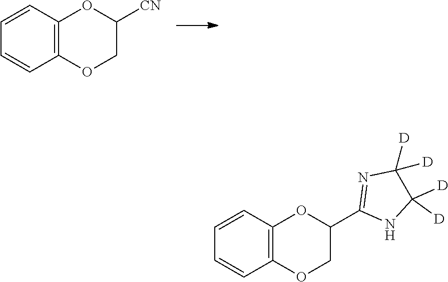 [SMILES: N#CC1COc2ccccc2O1 ; [2H]C1([2H])N=C(C2COc3ccccc3O2)NC1([2H])[2H]]

To a stirred mixture of 2,3-dihydro-1,4-benzodioxine-2-carbonitrile (120 mg, 0.75 mmol) in dry MeOH (2.5 mL) under an atmosphere of N₂ with ice bath cooling (0-5° C.) was added sodium methoxide (25% wt in MeOH) (6.8 μL, 0.03 mmol) in one portion. The mixture was warmed to rt and stirred for 6 h, then cooled in an ice bath and 1, 1,2,2-tetradeuterioethane-1,2-diamine (51 μL, 0.82 mmol) was added in one portion. After 5 mins, 1.25M HCl in MeOH (655 μL, 0.82 mmol) was added dropwise. The mixture was allowed to warm to rt and was stirred overnight, then diluted with H₂O (50 mL), basified with 33-34% aqueous NH₃ solution (3 mL) and extracted with DCM (CH₂Cl₂) (3×40 mL). The combined organic layers were dried over Na₂SO₄, filtered and the filtrate was concentrated under reduced pressure to afford the title compound (143 mg, 92%) as a solid. Retention time 0.982 mins; m/z=[M+H]⁺ calculated for C₁₁H₈D₄N₂O₂ 209.1; found 209.2; ¹H NMR (400 MHZ, CDCl₃) δ 6.98-6.85 (4H, m), 5.03 (1H, br. s), 4.89 (1H, dd, J=8.0, 2.8 Hz), 4.54 (1H, dd, J=11.6, 2.8 Hz), 4.18 (1H, dd, J=11.6, 8.0 Hz).

### Example 10: Enantiomerically Enriched Compounds

Compounds of the present disclosure (e.g., Compounds 1, 2, 3, 4, 49, 50, 51, 52 and 64) can be obtained as single enantiomers (e.g., as shown in the table below) e.g., by resolution of racemic deuterated compounds with (+)-tartaric acid or (−)-tartaric acid, or chiral HPLC.

| ID No. | Chemical Structure | IUPAC Name |
|---|---|---|
| 1-R | 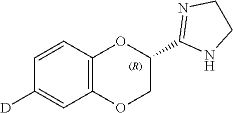 | (R)-2-(2,3-dihydrobenzo[b][1,4]dioxin-2-yl- 6-d)-4,5-dihydro-1H-imidazole |
| 1-S | 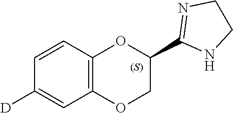 | (S)-2-(2,3-dihydrobenzo[b][1,4]dioxin-2-yl- 6-d)-4,5-dihydro-1H-imidazole |
| 2-R | 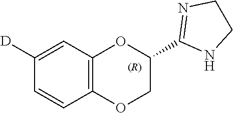 | (R)-2-(2,3-dihydrobenzo[b][1,4]dioxin-2-yl- 7-d)-4,5-dihydro-1H-imidazole |
| 2-S | 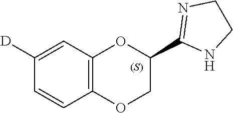 | (S)-2-(2,3-dihydrobenzo[b][1,4]dioxin-2-yl- 7-d)-4,5-dihydro-1H-imidazole |
| 3-R | 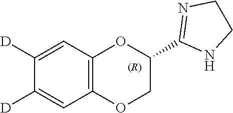 | (R)-2-(2,3-dihydrobenzo[b][1,4]dioxin-2-yl- 6,7-d2)-4,5-dihydro-1H-imidazole |
| 3-S | 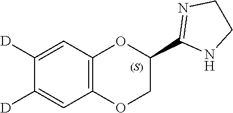 | (S)-2-(2,3-dihydrobenzo[b][1,4]dioxin-2-yl- 6,7-d2)-4,5-dihydro-1H-imidazole |
| 4-R | 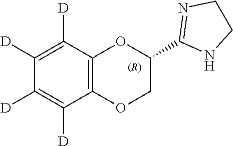 | (R)-2-(2,3-dihydrobenzo[b][1,4]dioxin-2-yl- 5,6,7,8-d4)-4,5-dihydro-1H-imidazole |
| 4-S | 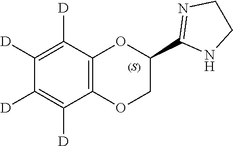 | (S)-2-(2,3-dihydrobenzo[b][1,4]dioxin-2-yl- 5,6,7,8-d4)-4,5-dihydro-1H-imidazole |
| 49-R | 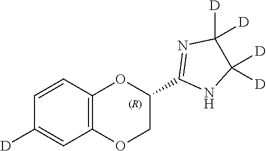 | (R)-2-(2,3-dihydrobenzo[b][1,4]dioxin-2-yl- 6-d)-4,5-dihydo-1H-imidazole-4,4,5,5-d4 |
| 49-S | 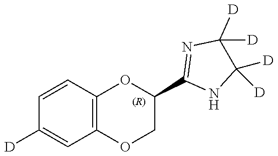 | (S)-2-(2,3-dihydrobenzo[b][1,4]dioxin-2-yl- 6-d)-4,5-dihydro-1H-imidazole-4,4,5,5-d4 |
| 50-R | 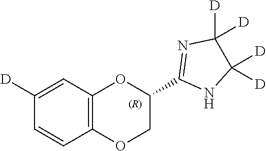 | (R)-2-(2,3-dihydrobenzo[b][1,4]dioxin-2-yl- 7-d)-4,5-dihydro-1H-imidazole-4,4,5,5-d4 |
| 50-S | 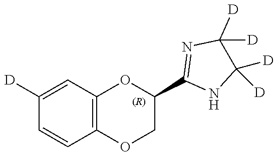 | (S)-2-(2,3-dihydrobenzo[b][1,4]dioxin-2-yl- 7-d)-4,5-dihydro-1H-imidazole-4,4,5,5-d4 |
| 51-R | 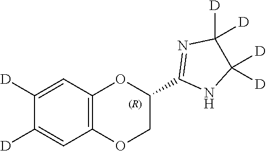 | (R)-2-(2,3-dihydrobenzo[b][1,4]dioxin-2-yl- 6,7-d2)-4,5-dihydro-1H-imidazole-4,4,4,5,5-d4 |
| 51-S | 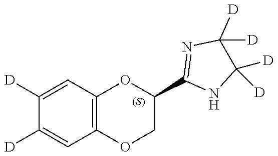 | (S)-2-(2,3-dihydrobenzo[b][1,4]dioxin-2-yl- 6,7-d2)-4,5-dihydro-1H-imidazole-4,4,5,5-d4 |
| 52-R | 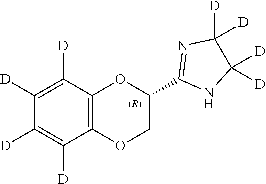 | (R)-2-(2,3-dihydrobenzo[b][1,4]dioxin-2-yl- 5,6,7,8-d4)-4,5-dihydro-1H-imidazole- 4,4,5,5-d4 |
| 52-S | 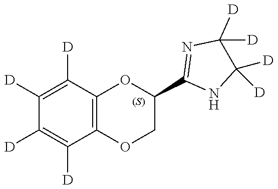 | (S)-2-(2,3-dihydrobenzo[b][1,4]dioxin-2-yl- 5,6,7,8-d4)-4,5-dihydro-1H-imidazole- 4,4,5,5-d4 |
| 64-R | 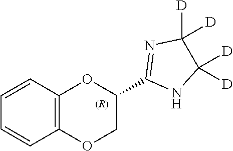 | (R)-2-(2,3-dihydrobenzo[b][1,4]dioxin-2-yl)- 4,5-dihydro-1H-imidazole-4,4,5,5-d4 |
| 64-S | 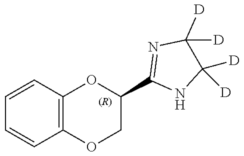 | (S)-2-(2,3-dihydrobenzo[b][1,4]dioxin-2-yl)- 4,5-dihydro-1H-imidazole-4,4,5,5-d4 |

### Biological Example 1: Pharmacokinetic Profile for the Deuterated Compounds

Stability in microsomes can be assessed using the protocol described in WO 2020/132461 which is incorporated by reference herein in its entirety. Stability in microsomes was assessed using the protocol described below.

### Evaluation of Metabolic Stability in Human Liver Microsomes

### Protocol Summary

Test compound (1 μM) was incubated with pooled liver microsomes. Test compound was incubated at 5 time points over the course of a 45 min assay and the test compound was analysed by LC-MS/MS.

### Objective

To determine the stability of the test compound in the presence of liver microsomes.

### Customer Requirements

Compound identifier, molecular weight and/or molecular formula.
50 μL of 10 mM test compound in DMSO per species per assay condition.

### Experimental Procedure

Pooled liver microsomes were purchased from a reputable commercial supplier. Microsomes were stored at −80° C. prior to use.

Microsomes (final protein concentration 0.5 mg/mL), 0.1 M phosphate buffer pH 7.4 and test compound (final substrate concentration 1 μM; final DMSO concentration 0.25%) were preincubated at 37° C. prior to the addition of NADPH (final concentration 1 mM) to initiate the reaction. A minus cofactor control incubation was included for each compound tested where 0.1 M phosphate buffer pH 7.4 was added instead of NADPH (minus NADPH). Two control compounds were included with each species. All incubations were performed singularly for each test compound.

Each compound was incubated for 0, 5, 15, 30 and 45 min. The control (minus NADPH) was incubated for 45 min only. The reactions were stopped by transferring incubate into acetonitrile at the appropriate time points, in a 1:3 ratio. The termination plates were centrifuged at 3,000 rpm for 20 min at 4° C. to precipitate the protein.

### Quantitative Analysis

Following protein precipitation, the sample supernatants were combined in cassettes of up to 4 compounds, internal standard was added and samples analysed using generic LCMS/MS conditions.

Optionally, if metabolite profiling was requested following the stability assay, a second assay was performed where the compound was incubated four times and the four resulting incubations were pooled to yield a higher sample concentration for analysis. The time point at which 30-70% of parent had degraded was then investigated at 3 different levels of metabolite profiling and/or identification.

### Data Analysis

From a plot of In peak area ratio (compound peak area/internal standard peak area) against time, the gradient of the line was determined. Subsequently, half-life and intrinsic clearance were calculated using the equations below:

$$Elimination rate constant ( k ) = ( - gradient )$$

$$\text{Half-life} ( t \frac{1}{2} ) ( min ) = 0.693 / k$$

$$Intrinsic clearance ( CLint ) ( μ L / min / mg protein ) = ( V \times 0.693 ) / t \frac{1}{2}$$

where V=Incubation volume (μL)/Microsomal protein (mg)

Relevant control compounds were assessed, ensuring intrinsic clearance values fell within the specified limits (if available). Any failures were rejected and the assay repeated. The intrinsic clearance (CLint) of the test compound and standard error (SE CLint), t½ and n (number of time points used to calculate CLint) were returned.

In these experiments, values equal to or more than a 15% increase in half-life were considered to be a significant difference if the apparent intrinsic clearance ratio (deuterated compound/idazoxan) was >1.15 or <0.85, then there was considered to be significant differentiation.

An alternative protocol is described in WO 2020/132461 (incorporated herein by reference in its entirety) and described briefly below.

Microsomal Assay: Human liver microsomes (20 mg/mL) are obtained from Xenotech, LLC (Lenexa, KS). β-nicotinamide adenine dinucleotide phosphate, reduced form (NADPH), magnesium chloride (MgCl₂), and dimethyl sulfoxide (DMSO) were purchased from Sigma-Aldrich.

Determination of Metabolic Stability: 7.5 mM stock solutions of each compound, or pharmaceutically acceptable salt thereof, are prepared in DMSO. The 7.5 mM stock solutions were diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 pL aliquot of the test compound (12.5-50 μM) is added to the microsomes and the mixture was pre-warmed for 10 minutes. Reactions are initiated by addition of pre-warmed NADPH solution. The final reaction volume is 0.5 mL and contains 4.0 mg/mL human liver microsomes, 0.25 μM test compound, and 2 mM NADPH in 0.1 M potassium phosphate buffer, pH 7.4, and 3 mM MgCl₂. The reaction mixtures are incubated at 37° C., and 50 pL aliquots are removed at 0, 5, 10, 20, and 30 minutes and added to shallow-well 96-well plates which contain 50 pL of ice-cold ACN (acetonitrile) with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart and the positive control, 7-ethoxycoumarin (1 μM). Testing is done in triplicate.

Data analysis: The in vitro T_{1/2}s for test compounds were calculated from the slopes of the linear regression of % parent remaining (In) vs incubation time relationship. in vitro T_{1/2}=0.693/k

k=−[slope of linear regression of % parent remaining (In) vs incubation time]

The apparent intrinsic clearance is calculated using the following equation:

$${CL}_{int} ( \text{mL/min/kg} ) = ( 0.693 / in vitro T ) ( \text{Incubation Volume/mg of microsomes} ) ( 45 mg microsomes / gram of liver ) ( 20 gm of liver / kg b . w . )$$

Data analysis was performed using Microsoft Excel Software.

### Evaluation of Metabolic Stability in Human Liver Microsomes—Results

Test Compound Concentration=3 μM
Microsome Concentration=0.5 mg/mL
Time Points=0, 5, 15, 30, 45 minutes
Cofactor=NADPH
Final DMSO Concentration=0.25%
Controls=0 μM (blank); Positive control compounds with known activity
Analysis method=LC-MS/MS

TABLE 2
Metabolic stability in human liver microsomes
of representative deuterated compounds.

| | CLᵢₙₜ (μL/min/mg | t_{1/2} | |
|---|---|---|---|
| Compound ID | protein) | (min) | n |
| Compound 1 | 2.87 | 483 | 5 |
| Compound 2 | 1.06 | 1310 | 5 |
| Compound 3 | 2.23 | 622 | 5 |
| Compound 4 | 2.59 | 536 | 5 |
| Compound 49 | 1.49 | 932 | 5 |
| Compound 50 | 0.0253 | 54800 | 5 |
| Compound 51 | 1.68 | 825 | 5 |
| Compound 52 | 2.98 | 465 | 5 |
| Compound 64 | 2.91 | 477 | 5 |
| Idazoxan | 5.39 | 257 | 5 |
| Dextromethorphan | 48.1 | 28.8 | 5 |
| Verapamil * | 315 | 4.40 | 3 |
| Diazepam | 89.4 | 15.5 | 5 |
| Diphenhydramine * | 690 | 2.01 | 3 |

* 30 and 45 minute time points excluded because verapamil and diphenhydramine are metabolized very fast and are completely gone by 30 min. Thus, the 30 min and 45 min timepoints were not able to be measured.

Based on the results in Table 2, Compounds 1, 2, 3, 4, 49, 50, 51, 52 and 64 exhibited significant differences in in vitro clearance and half-life compared to idazoxan.

### Biological Example 2: Oral Bioavailability in Rats

Pharmacokinetics of test compounds are determined following a single intravenous or oral administration in rats. A pharmacokinetic (PK) study is performed in three male Sprague-Dawley (SD) rats following intravenous (IV) and oral (PO) administration of idazoxan, or deuterated-idazoxan analog of the present disclosure, at 1 mg/kg (IV) and 10 (PO) mg/kg. Test compounds, or idazoxan, are measured in plasma.

### Rat Strain

Rats used in these studies are supplied by Charles River (Margate UK) and are specific pathogen free. The strain of rats is Sprague Dawley. Male rats are 175-225 g on receipt and are allowed to acclimatise for 5-7 days.

### Animal Housing

Rats are group housed in sterilised individual ventilated cages that expose the animals at all times to HEPA filtered sterile air. Animals have free access to food and water (sterile) and have sterile aspen chip bedding (at least once weekly).

The room temperature is 22° C.+/−1° C., with a relative humidity of 60% and maximum background noise of 56 dB. Rats are exposed to 12-hour light/dark cycles.

### Treatment

Each test compound is diluted 10% v/v DMSO, 40% v/v PEG-400, 50% v/v water. The test compounds are each administered in a dose volume of 2 mL/kg for intravenous (IV) and 5 mL/kg (PO) for oral routes of administration.

### Single IV PO Dose Pharmacokinetics Study in Rats

Each test compound is administered as a single IV bolus (via a lateral tail-vein) or a single oral gavage in cohorts of 3 rats per route. Following dose administrations, a 100 μL whole blood sample (EDTA) is collected via the tail-vein at time-points described in Table 3. The blood is centrifuged to separate plasma. Approximately 40 μL of plasma is dispensed per time-point, per rat, in a 96 well plate and frozen until analysis. Bioanalysis is carried out on plasma samples.

TABLE 3
Single IV and oral dose pharmacokinetics
profile of test articles in rat plasma

| | | | | | No. |
|---|---|---|---|---|---|
| | | | Dose | Blood sample collection | of |
| Group | Test article | Route | (mg/kg) | (post dose) | rats |
| 1 | Idazoxan | IV | 1 | 5 min, 15 min, 30 min, 1 | 3 |
| | | | | h, 2 h, 4 h, 7 h, 24 h | |
| 2 | Idazoxan | PO | 10 | 15 min, 30 min, 45 min, | 3 |
| | | | | 1 h, 2 h, 4 h, 7 h, 24 h | |
| 3 | Deuterated | IV | 1 | 5 min, 15 min, 30 min, 1 | 3 |
| | Idazoxan | | | h, 2 h, 4 h, 7 h, 24 h | |
| | Analog | | | | |
| 4 | Deuterated | PO | 10 | 15 min, 30 min, 45 min, | 3 |
| | Idazoxan | | | 1 h, 2 h, 4 h, 7 h, 24 h | |
| | Analog | | | | |

### EQUIVALENTS

Those skilled in the art will recognize, or be able to ascertain using no more than routine experimentation, many equivalents to the specific embodiments and methods described herein. Such equivalents are intended to be encompassed by the scope of the present application.

All patents, patent applications, and literature references cited herein are hereby expressly incorporated by reference.

What is claimed is:

1. A compound represented by Formula (I), 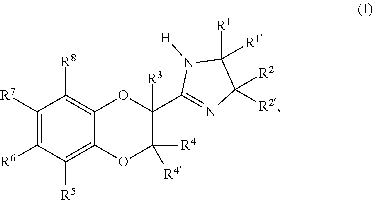
or a pharmaceutically acceptable salt, solvate, hydrate, tautomer, or stereoisomer thereof, wherein R¹, R¹′, R², R²′, R³, R⁴, R⁴′, R⁵, R⁶, R⁷, and R⁸ are each independently selected from hydrogen and deuterium, provided that at least one of R¹, R¹′, R², R²′, R³, R⁴, R⁴′, R⁵, R⁶, R⁷, and R⁸ is deuterium.

2. A compound represented by Formula (II) or Formula (III), 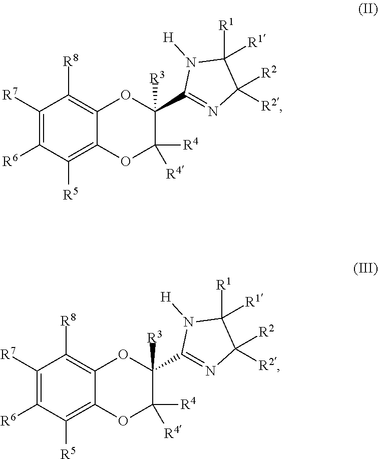
or a pharmaceutically acceptable salt, solvate, hydrate, tautomer, or stereoisomer thereof, wherein R¹, R¹′, R², R²′, R³, R⁴, R⁴′, R⁵, R⁶, R⁷, and R⁸ are each independently selected from hydrogen and deuterium, provided that at least one of R¹, R¹′, R², R²′, R³, R⁴, R⁴′, R⁵, R⁶, R⁷, and R⁸ is deuterium.

3. A compound represented by Formula (II-A) or Formula (III-A), 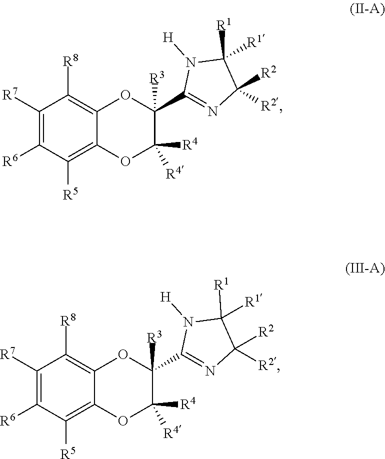
or a pharmaceutically acceptable salt, solvate, hydrate, tautomer, or stereoisomer thereof, wherein R¹, R¹′, R², R²′, R³, R⁴, R⁴′, R⁵, R⁶, R⁷, and R⁸ are each independently selected from hydrogen and deuterium, provided that at least one of R¹, R¹′, R², R²′, R³, R⁴, R⁴′, R⁵, R⁶, R⁷, and R⁸ is deuterium.

4. The compound according to claims 1-3, wherein at least one of R⁵, R⁶, R⁷ and R⁸ is deuterium.

5. The compound according to claims 1-4, wherein at least two of R⁵, R⁶, R⁷ and R⁸ are deuterium.

6. The compound according to claims 1-4, wherein at least three of R⁵, R⁶, R⁷ and R⁸ are deuterium.

7. The compound according to claims 1-4, wherein all of R⁵, R⁶, R⁷ and R⁸ are deuterium.

8. The compound according to claims 1-7, wherein at least one of R¹, R¹′, R², R²′ is deuterium.

9. The compound according to claims 1-7, wherein at least two of R¹, R¹′, R², R²′ are deuterium.

10. The compound according to claims 1-7, wherein at least three of R¹, R¹′, R², R²′ are deuterium.

11. The compound according to claims 1-7, wherein all of R¹, R¹′, R², R²′ are deuterium.

12. The compound of either claim 1 or 2, wherein the compound is represented by Formula (I-A) 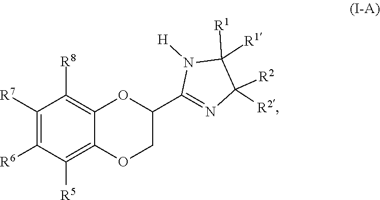
or a pharmaceutically acceptable salt, solvate, hydrate, tautomer, or stereoisomer thereof, wherein R¹, R¹′, R², R²′, R⁵, R⁶, R⁷, and R⁸ are each independently selected from hydrogen and deuterium, provided that at least one of R¹, R¹′, R², R²′, R⁵, R⁶, R⁷, and R⁸ is deuterium.

13. The compound according to claim 12, wherein at least one of R¹, R¹′, R², R²′ is deuterium.

14. The compound according to either claim 12 or 13, wherein at least one of R¹ and R² is deuterium.

15. The compound according to any one of claims 12-14, wherein at least one of R⁵, R⁶, R⁷, and R⁸ is deuterium.

16. The compound according to any one of claims 12-14, wherein at least one of R⁶, and R⁷ is deuterium.

17. The compound of any one of claim 1-3 or 12 selected from the list consisting of: 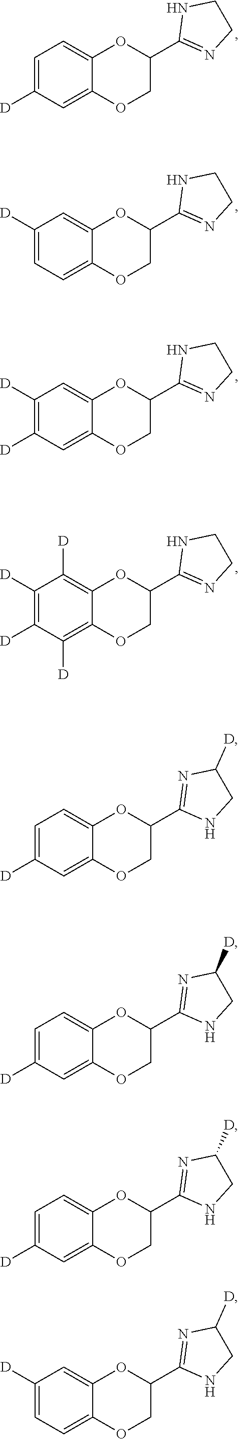 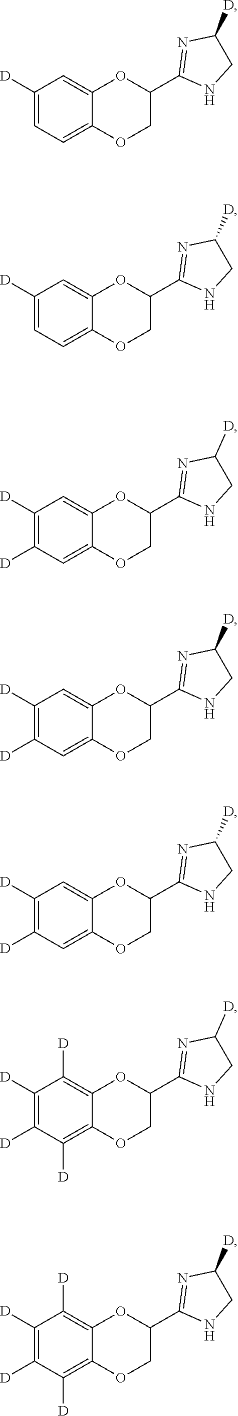 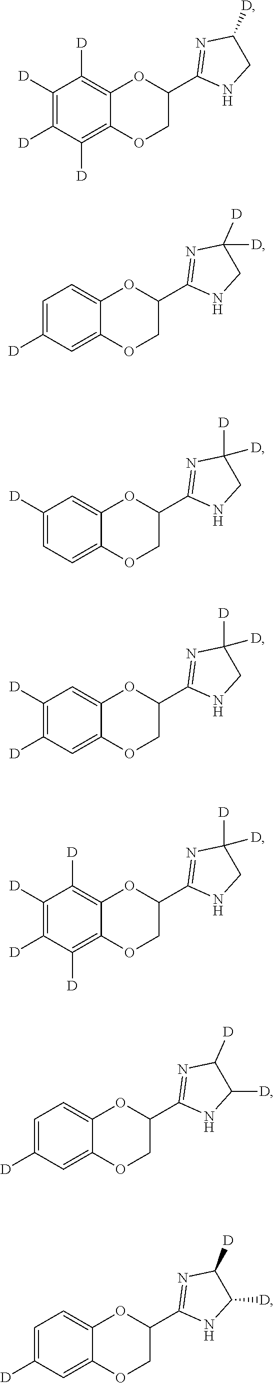 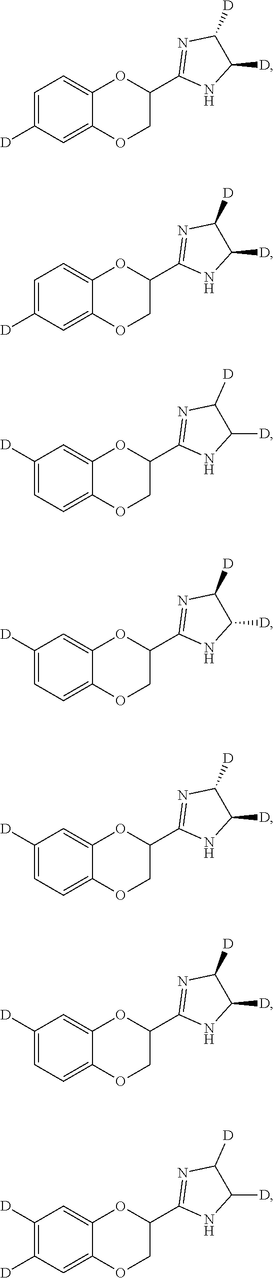 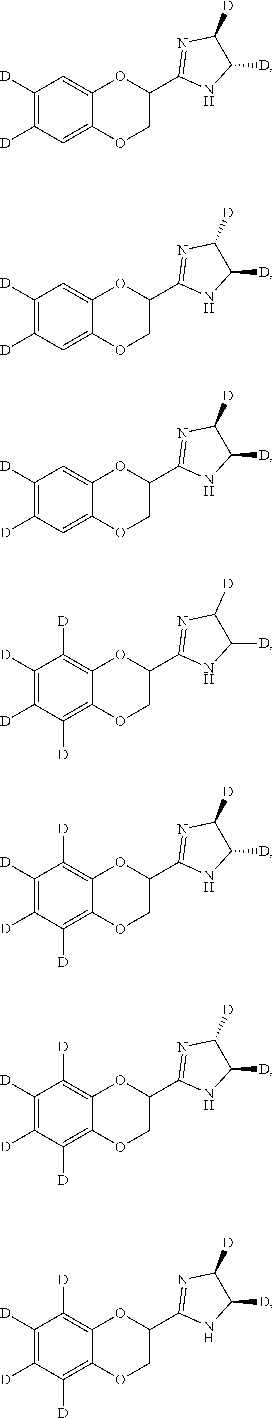 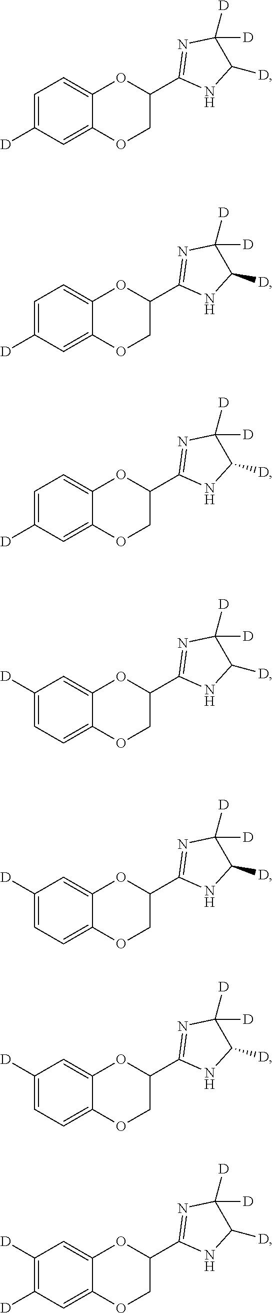 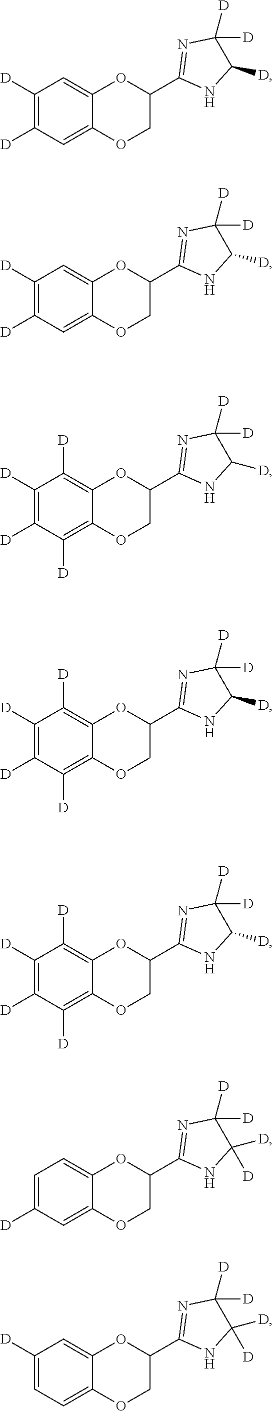 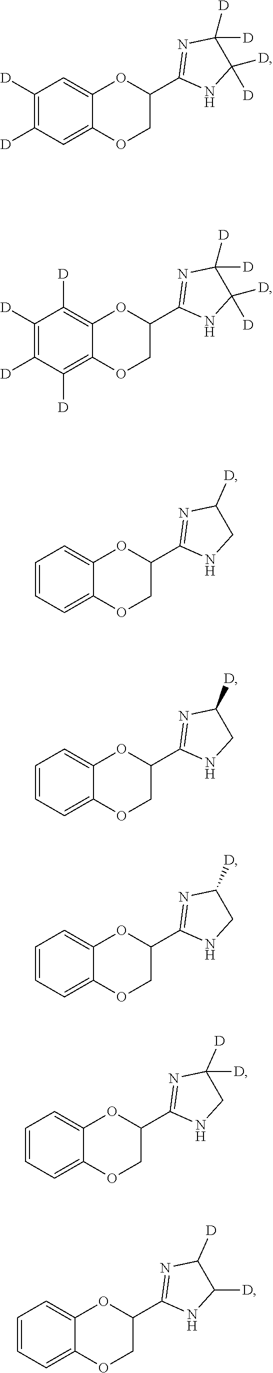 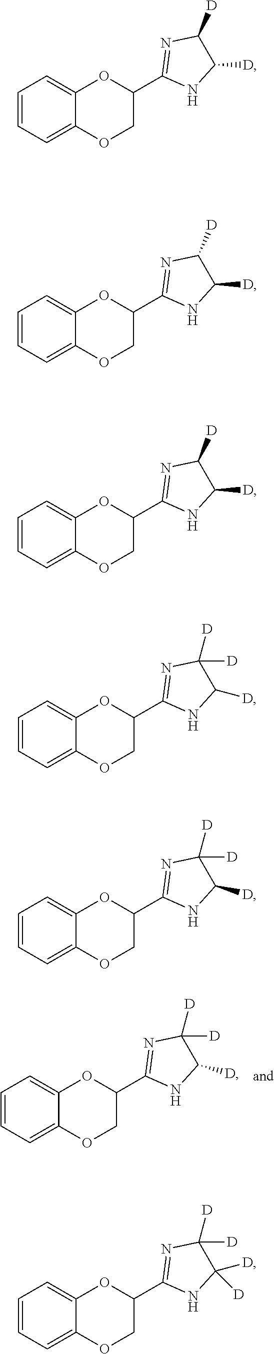
or a pharmaceutically acceptable salt, solvate, hydrate, tautomer, or stereoisomer thereof.

18. The compound of any one of claim 1-3 or 12 selected from the list consisting of: 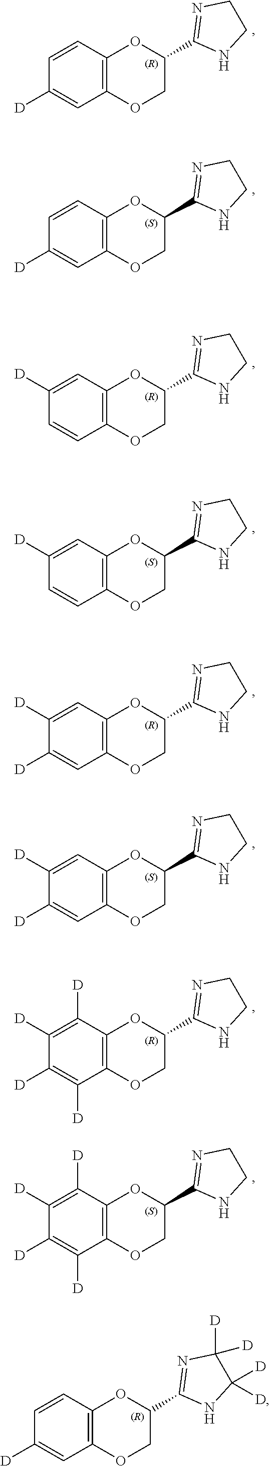 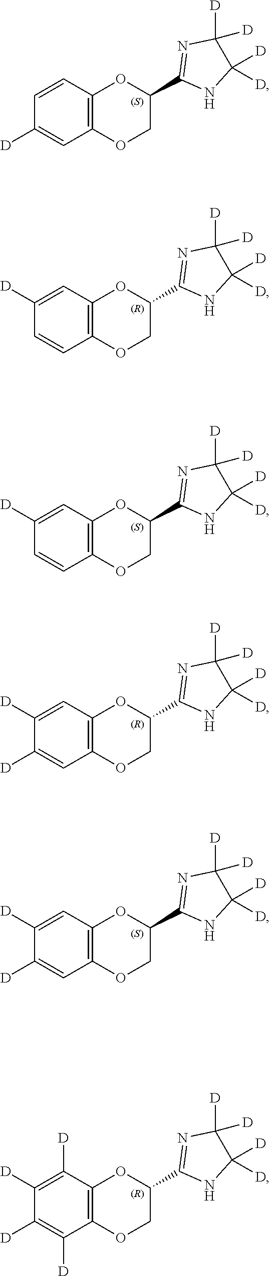 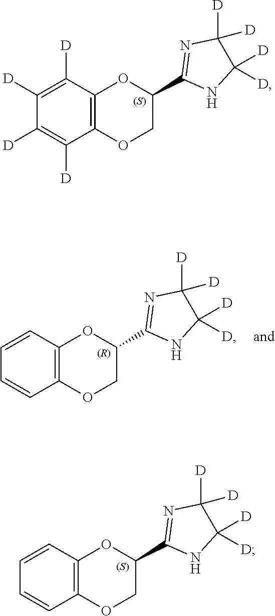
or a pharmaceutically acceptable salt, solvate, hydrate, tautomer, or stereoisomer thereof.

19. The compound of any one of the preceding claims, wherein each position designated specifically as deuterium has at least 90% incorporation of deuterium.

20. The compound of any one of the preceding claims, wherein each position designated specifically as deuterium has at least 95% incorporation of deuterium.

21. The compound of any one of the preceding claims, wherein each position designated specifically as deuterium has at least 97% incorporation of deuterium.

22. A pharmaceutical composition comprising a compound of any one of the preceding claims and a pharmaceutically acceptable excipient.

23. A method of treating or preventing a disease or condition selected from psychosis, schizophrenia, schizoaffective disorder, Parkinson's disease, Lewy body dementia, sleep disorder, agitation, mood disorder, thromboembolic disorder, autism, attention deficit hyperactivity disorder, and any combination thereof, in a subject in need thereof, comprising administering to the subject an effective amount of the compound according to any one of claims 1-21 or a pharmaceutical composition of claim 22.

24. Use of a compound of any one of claims 1-21, or a pharmaceutically acceptable salt thereof, for the manufacture of a medicament for treating or preventing a disease or condition selected from psychosis, schizophrenia, schizoaffective disorder, Parkinson's disease, Lewy body dementia, sleep disorder, agitation, mood disorder, thromboembolic disorder, autism, attention deficit hyperactivity disorder, and any combination thereof.

25. A kit comprising the compound of any one of claims 1-21, or the pharmaceutical composition of claim 22, with instructions for the use thereof.
